(19) United States
(12) Patent Application Publication
(10) Pub. No.: US 2023/0301905 A1
(43) Pub. Date: Sep. 28, 2023

(54) INHALABLE THERAPEUTIC AGENTS

(71) Applicant: Cila Therapeutic Inc., Jamaica Plain, MA (US)

(72) Inventors: Safia RIZVI, Jamaica Plain, MA (US); Dennis UNDERWOOD, Jamaica Plain, MA (US)

(21) Appl. No.: 18/023,686

(22) PCT Filed: Aug. 26, 2021
(86) PCT No.: PCT/US2021/047774

§ 371 Date: Feb. 27, 2023

Related U.S. Application Data
(60) Provisional application No. 63/070,718, filed on Aug. 26, 2020.

(51) Int. Cl. A61K 9/00; A61K 31/185; A61K 38/46; A61K 38/13; A61K 31/138; A61K 31/538; A61K 31/167; A61K 31/473; A61K 31/56; A61K 9/14

(52) U.S. Cl. CPC A61K 9/0075; A61K 9/14; A61K 31/138; A61K 31/167; A61K 31/185; A61K 31/473; A61K 31/538; A61K 31/56; A61K 38/13; A61K 38/465

(57) ABSTRACT

Described herein are compositions and methods of use thereof. The compositions include a first population of inhalable particles and a second population of inhalable particles, wherein the first inhalable particles include a first mucolytic contained within a first biodegradable encapsulation, and the second population of inhalable particles includes the first or a second mucolytic contained within a second biodegradable encapsulation.

### BACKGROUND OF THE INVENTION

Without limiting the scope of the invention, its background is described in connection with inhalable therapeutic agents. More particularly, the invention describes inhalable particles comprising a mucolytic contained within a biodegradable encapsulation such as a liposomal, nanoparticle, microsphere, or engineered particle formulation for the treatment of lung, airway, and respiratory diseases.

The main function of the respiratory system is to draw air into the lungs of a subject and allow the exchange of gases with blood circulating and perfusing the lung tissues. The lungs are the body’s most important respiratory organ where normal gaseous exchange of essential oxygen and carbon dioxide as well as carbon monoxide are carried out effectively and efficiently. The lungs preform its gaseous exchange function as it has direct access and exposure to the outside air and environment, with which this gaseous exchange is carried out. To achieve this essential role the epithelial lung tissues are kept moist with a thin film of hydrated mucus, which include various proteins, including mucin, physiological cations and anions such as sodium, potassium, calcium, chloride, bicarbonate, carbonates, phosphate etc., that are maintained in aqueous osmolar concentrations, in addition to other proteins and enzymes.

Mucus is normal product of tracheobronchial tree and serves as the first line of defense for respiratory apparatus. The mucus transport system is the fundamental defense of the airways against inhaled debris, bacteria, viruses, and other foreign agents. Inhaled foreign particles are trapped in the mucus layer and subsequently propelled out of the lungs via mucociliary clearance. The well hydrated mucus facilitates this ciliary clearance of these foreign agents and keeps the respiratory tract protected. Mucociliary clearance (MCC) in healthy and normal lungs is facilitated by the movement of cilia, that clear well hydrated mucus/liquid, and is also facilitated by cellular ion transport system, such as the sodium and chloride transport system. In the absence of sufficient mucus hydration and functioning cilia and ion transport systems, the mucus becomes excessively viscous and adherent forming “mucus plugs”. The composition of mucus plugs vary for different diseases but the main component of mucus plugs are always polymerized mucus glycoproteins, mucin, extracellular DNA, neutrophils, and filamentous actin. Several diseases of the respiratory tract are characterized by this unusually thick and adhesive forms of mucus or mucus plugs This results in airway obstruction, inability to breath properly (insufficient oxygen exchange), and recurrent lung, airway, and ear infections. Recurrent infection causes permanent lung damage and may lead to respiratory failure.

These muco-obstructive respiratory diseases, including Cystic Fibrosis, Primary Ciliary Dyskinesia, Bronchiectasis, chronic obstructive pulmonary disease (COPD), Rhinitis, Rhinosinusitis and more, affect the lives of > 30 M people in the US. Furthermore, pathological mucus plugs have also been found in the lungs of patients with severe COVID-19 viral infection. These plugs are part of COVID-19 sequalae and contribute to acute respiratory distress.

The origin and compositions of these pathological mucus plugs is diverse in different disease. The initial cause of the various respiratory diseases may vary and include environmental exposure to various compounds, free radicals, reactive oxygen species (ROS), and others (due to smoking, toxin exposure, chronic respiratory infection etc.), as well as genetic etiologies such as mutation in functional proteins in Cystic Fibrosis (CF) and Primary Ciliary Dyskinesia (PCD). In Cystic Fibrosis, the specific genetic mutation results in high concentrations of DNA from degenerating polymorphonuclear leucocytes in the lung. This excess of extracellular DNA increases the viscosity of sputum and makes it more difficult to clear the sputum from the respiratory tract. This increases the susceptibility to infection which, in turn, provokes an inflammatory response. The cycle of infection, inflammation and obstruction leads to progressive destruction of lung tissue and reduced life expectancy.

In the various respiratory and lung diseases, it is widely established that a common manifestation of these diseases is a lack of, or diminished, normal mucus clearance and formation of thick, viscous, and adhesive mucus or sputum. Increasingly highly viscous and adherent mucus or sputum is considered to be caused by an increase of chronic “oxidative stress,” lack of hydration and movement of mucus and results in an altered state of mucus, a common element of which is oligomerization of normal mucus proteins such as mucin and other proteins via multiple inter- and intra-molecular disulfide bond formation. This is coupled with an increased non-covalent protein-protein interaction that further aggregates the protein(s) polymer structures due to increased hydrogen-bonding, and hydrophilic, hydrophobic, and inter- and intra-protein interactions. These molecular changes/interactions thereby increase the elasticity and viscosity of the naturally maintained mucus and result in a decreasing efficiency of the natural mucus clearance mechanisms and system. The mucus clearance is also hampered by dysfunction in cilia (tiny hair like structure that line the respiratory apparatus) and ion transport proteins caused by genetic mutations in relevant proteins. Collectively, the resulting inter- and intra-protein disulfide bonds and various hydrogen bonding, protein-protein hydrophilic and hydrophobic interactions are responsible for altering the normal mucus structure and building and sustaining the protein oligomerization resulting in mesh-like protein networks commonly known as “mucus plugs.” Furthermore, inflammatory processes, including recruitment of neutrophils to the site of inflammation, toxic exposure or infection result in an increased oxidative environment with concomitant cell death, this in turn accumulates cellular debris including DNA, lipids, fibrin and foreign particles and pathogens leading to thick mucus that is too viscous and adhesive for normal flow and removal. The decreased MCC in turn serves to exacerbate the mucus plugs and provide a fertile growth environment for respiratory bacterial growth. This cascade of molecular processes cumulatively leads to chronic breathing problems, recurrent airway, lung, and nasal infections with persistence and progression toward lung damage.

Regardless of underlying origin of their source the accumulated mucus, it is the resulting mucus plugs that become the principal cause of common respiratory problems including diminished muco-ciliary clearance, airway blockage, recurrent infections and respiratory distress. Furthermore, these mucus plugs harbor pathogens and promote recurrent lung infections, irreversible lung damage, long-term complications and disease progression that can lead to respiratory failure.

Altered-structured and highly viscous mucus plug are key manifestation of multiple pulmonary disease such as primary ciliary disease (PCD), cystic fibrosis, bronchiectasis, sinusitis, rhinosinusitis, and chronic obstructive pulmonary disease (COPD, an umbrella term used for a broad array of pulmonary disorders such as emphysema, bronchitis etc.). Aggregated-structured mucus or sputum has altered macromolecular composition and biophysical properties that vary with disease. The underlying causes of these diseases vary based on genetic and environmental factors, but common features in all these diseases are diminished mucus clearance, resulting in airway obstruction, entrapment of bacteria and pathogen, recurrent infection and repeated inflammation. This in turn progressively damages the lungs and can eventually cause respiratory failure or/and need for lung transplant.

Patients of muco-obstructive diseases live with the risk of exacerbation episodes of respiratory distress that frequently lead to emergency room visits and hospitalizations. Exacerbations create a huge economic burden on families and the health care system and negatively impact their quality of life. Current standard of care for most of these diseases (with the exception of CF) remains focused on addressing symptoms and complications. These comprise:
control, and treat infections of the lungs, sinus, and ear (oral and IV antibiotics and anti-infectives)
hydrate and remove the trapped viscous mucus and sputum (moisture inhalers and saline bronchial lavage)
reduce swelling and Inflammation (corticosteroids and bronchodilators)
expand airways to facilitate breathing (bronchodilators)
CPT (chest physical therapy) manually or with the assistance of devices
surgery (tympanostomy, sinus surgery, and lung transplant)
hydration with osmotic agents such as hypertonic saline and mannitol

All of these methods, with the exception of surgery, show only marginal or temporary relief and do not effectively address the basic problem of thick, viscous, adhesive mucus plugs that are the main culprit, causing breathing difficulties, trapped cellular and particulate debris and pathogens, recurrent infections, and irreversible and progressive lung damage.

A safe, effective, and dependable therapeutic option such as the composition described in this invention for disrupting the critical bonds responsible for polymerization of protein/the formation of mucus plugs and thus fluidification of viscous mucus is therefore much needed to address and alleviate the basic issue in many rare and common lung and respiratory/pulmonary diseases and facilitate MCC. This would significantly improve the quality of life for patients with the above-mentioned diseases. It would first, fluidify mucus and clear the airways obstruction, reduce oxygenation distance, improve gaseous exchange and address breathing problems, second, prevent recurring infection by completely exposing pathogens (previously shielded in mucus plugs) to antibiotics, thus rendering other intervention more effective and, enabling corticosteroids and bronchodilators to reach their target location without being blocked by thick mucus layers and third, prevent permanent lung damage.

Certain mucolytics have been used in a nebulized solution form in very high doses in a postoperative ICU setting (400 mg-4 g/day). One of the two approved prescription mucolytics is recombinant human deoxyribonuclease or rhDNAse that is indicated in conjunction with standard therapies for the management of CF patients to improve pulmonary function. In CF patients with an FVC ≥ 40% of predicted, daily administration of rhDNAse has also been shown to reduce the risk of respiratory tract infections requiring parenteral antibiotics.

Mechanistically, rhDNAse is an enzyme that selectively cleaves DNA. For Cystic fibrosis patients, DNase catalyzes the hydrolytic cleavage of phosphodiester linkages in the in the DNA backbone of extracellular DNA in the mucus thus degrading DNA. Therefore, decreases the concentration of DNA and reduce the viscoelastic properties of sputum in CF patients which results in an improved lung function.

Currently available mucolytic PULMOZYNE®, is a sterile, clear, colorless, highly purified solution in single-use ampules. Each ampule delivers 2.5 mL of the solution to the nebulizer bowl. Each mL of aqueous solution contains 1 mg dornase alfa, calcium chloride dihydrate (0.15 mg) and sodium chloride (8.77 mg) and sterile water. The solution contains no preservative. The nominal pH of the solution is 6.3. It is administered by inhalation of an aerosolized mist of DNase solution produced by a compressed air driven nebulizer system with an adequate air flow and equipped with a mouthpiece or suitable face mask. While rhDNAse/ PULMOZYNE®, and has been effective in improving the lung function, reducing rate of infection and improving the quality of life for Cystic Fibrosis patients, it has not been approved for other muco-obstructive disease. Doctors do utilize it for other diseases on a case-by-case basis.

PULMOZYNE®, has several limitations. PULMOZYNE®, has low deposition efficiency, reported in the range between 7.8% and 31.2%. PULMOZYNE®, also has storage restrictions, including the need to be stored in a protective foil pouch under refrigeration. A dose of PULMOZYNE®, takes 20-40 minutes to administer. Further, the nebulizer cup must be rinsed with warm water, cleaned of excess liquid, and allowed to air dry following each use. Further cleaning may be required, such as washing the nebulizer cup, mouthpiece, and/or mask with soap and/or disinfectant. Further, while the drug may be safe at these doses in ICU, it is not suitable for chronic out-patient use. Therefore, it has never been utilized and approved for chronic out-patient use.

The disadvantages of PULMOZYNE®, reduce effectiveness and patient adherence to the treatment, jeopardizing the rhDNAse therapy. This molecule has the propensity to readily form dimers and lose its mucolytic activity. Therefore, without stabilizing the activity and using an appropriate delivery method, only a fraction of the dose makes it to the lung. Furthermore, the particle size, flight characteristics, and delivery method have impact on the level of penetration of the drug into the airways and lungs.

Therefore, it is important to formulate and deliver the molecule in a way to stabilize the active form and ensure deeper penetration into the lungs and convenient delivery of a significant percentage, e.g., 60%, of the dose to patient airways.

There is also a desire to protect some mucolytic from degradation or loss of activity before being delivered to the treatment site. Certain mucolytic agents can readily oxidize before reaching their target, which may diminish their efficacy. This may explain inconsistent results in clinical trials. The need exists to provide protection from degradation or loss of activity for a mucolytic in order to maximize its potency.

### SUMMARY OF THE INVENTION

Accordingly, it is an object of the present invention to overcome these and other drawbacks of the prior art by providing novel inhalable therapeutic agents that allow effective dissolution of mucus.

It is another object of the present invention to provide novel inhalable therapeutic agents designed to provide effective treatment at lower doses of the drug so as to reduce possible side effects and allow for self-administration in repeated applications performed multiple times.

It is a further object of the present invention to provide novel inhalable therapeutic agents configured for administration either by a medical professional or self-administration so as to make such treatment suitable for out-patient delivery.

It is yet another object of the present invention to provide novel therapeutic agents configured to protect the active drug from degradation and prevent loss of mucolytic activity prior to its direct delivery at the target site of the airways, whereby preserving its potency until its immediate interaction at the target site. In the most basic form, the present invention may be described as at least one or several pluralities of inhalable particles. Each particle may include one or more mucolytic or another suitable active therapeutic agent contained within one or several different types of biodegradable encapsulations, desirably without the risk of pre-delivery interaction. The number and respective concentration of therapeutic agents including at least one mucolytic, the type of biodegradable encapsulation selected for each therapeutic agent, and the size of inhalable particles may be adjusted depending on a particular type of airways disorder in order to deliver the intended medication to an intended location of the airways to effectively treat the underlying condition.

In embodiments, at least two types and/or sizes of inhalable particles may be enclosed in a single dispersing apparatus configured for self-administration by the subject in repeated applications based on a predetermined schedule. At least one of such inhalable particles may include a mucolytic within a biodegradable encapsulation, such as a liposomal formulation, microsphere, nanoparticles or engineered spray-dried particles. Another embodiment includes two such inhalable particles of the same therapeutic, only of different sizes.

Exemplary embodiments of the invention are described in the enumerated paragraphs below.

E1. A composition comprising a population of inhalable particles, wherein the particles comprise a first mucolytic contained in a biodegradable lipid encapsulation, and wherein the composition is for delivery via a dispersing apparatus.

E2. The composition of E1, wherein the is selected from a group of compounds consisting of: sodium 2-mercaptoethane sulfonate, N-acetylcysteine, L-α-ureido-mercaptopropionic acid, Bromhexine, ascorbic acid, vitamin E, tris(2-carboxyethyl)phosphine hydrochloride, N-butylcysteine, reduced glutathione, N-derivatives and C-derivatives of amino acid cysteine, di-peptide of cysteine and glutamic acid, di-peptide of aspartic acid, Ambroxol Hydrochloride, DNAse, and a DNA cleaving agent.

E3. The composition as in E1, wherein the first mucolytic is 2-mercaptoethane sulfonate (e.g., sodium 2-mercaptoethane sulfonate).

E4. The composition as in E1, wherein the mucolytic is N-acetylcysteine.

E5. The composition as in E1, wherein the mucolytic is L-α-ureido-mercaptopropionic acid.

E6. The composition as in E1, wherein the mucolytic is Bromhexine.

E7. The composition as in E1, wherein the mucolytic is ascorbic acid.

E8. The composition as in E1, wherein the mucolytic is vitamin E.

E9. The composition as in E1, wherein the mucolytic is tris(2-carboxyethyl)phosphine hydrochloride.

E10. The composition as in E1, wherein the mucolytic is N-butylcysteine.

E11. The composition as in E1, wherein the mucolytic is reduced glutathione.

E12. The composition as in E1, wherein the mucolytic is a N-derivative of amino acid cysteine.

E13. The composition as in E1, wherein the mucolytic is a C-derivative of amino acid cysteine.

E14. The composition as in E1, wherein the mucolytic is a di-peptide of cysteine and glutamic acid.

E15. The composition as in E1, wherein the first mucolytic is a di-peptide of aspartic acid.

E16. The composition as in E1, wherein the mucolytic is Ambroxol Hydrochloride.

E17. The composition as in E1, wherein the mucolytic is a DNAse.

E18. The composition as in E1, wherein the mucolytic is a DNA cleaving agent.

E19. The composition as in any one of E1 to E18, wherein the population of inhalable particles are sized to be from about 0.01 microns to about 49 microns, whereby the population of inhalable particles are configured for absorption at a target location of the airways of a subject.

E20. The composition as in E19, wherein the target location of the airways of the subject is the upper respiratory tract or the lower respiratory tract.

E21. The composition as in E19 or E20 further comprising additional particles of mucolytics contained within biodegradable encapsulations, the additional particles are sized for predominant absorption at additional locations of the airways upon inhalation of the composition by the subject.

E22. The composition as in any one of E19 to E21, wherein the biodegradable encapsulation is configured to release the mucolytic immediately upon inhalation of the composition by the subject.

E23. The composition as in E22 further comprising additional particles comprising a mucolytic contained within a biodegradable encapsulation configured to release the mucolytic with additional predetermined delays.

E24. The composition as in any one of E1 to E23, wherein the inhalable particles are stored in a solution form or a dry powder form.

E25. The composition as in any one of E1 to E24, wherein the inhalable particles are stored in a solution form.

E26. The composition as in any one of E1 to E24, wherein the inhalable particles are stored in a dry form.

E27. The composition as in any one of E1 to E26, wherein the dispersing apparatus is configured upon activation to provide each dose of the composition containing from about 5 mg to about 200 milligrams of the first mucolytic.

E28. The composition as in any one of E1 to E27, wherein the dispersing apparatus is a dry-powder inhaler.

E29. A composition comprising a first population of inhalable particles and a second population of inhalable particles, wherein the first inhalable particles comprises a first mucolytic contained within a first biodegradable encapsulation, the second population of inhalable particles comprises the first or a second mucolytic contained within a second biodegradable encapsulation.

E30. The composition as in E29, wherein the first mucolytic or the second mucolytic are selected from a group of compounds consisting of: sodium 2-mercaptoethane sulfonate, N-acetylcysteine, L-α-ureido-mercaptopropionic acid, Bromhexine, ascorbic acid, vitamin E, tris(2-carboxyethyl)phosphine hydrochloride, N-butylcysteine, reduced glutathione, N-derivatives and C-derivatives of amino acid cysteine, di-peptide of cysteine and glutamic acid, di-peptide of aspartic acid, Ambroxol Hydrochloride, DNAse, and a DNA cleaving agent.

E31. The composition as in E29 or E30, wherein the first mucolytic is 2-mercaptoethane sulfonate (e.g., sodium 2-mercaptoethane sulfonate).

E32. The composition as in E29 or E30, wherein the first mucolytic is N-acetylcysteine.

E33. The composition as in E29 or E30, wherein the first mucolytic is L-α-ureido-mercaptopropionic acid.

E34. The composition as in E29 or E30, wherein the first mucolytic is Bromhexine.

E35. The composition as in E29 or E30, wherein the first mucolytic is ascorbic acid.

E36. The composition as in E29 or E30, wherein the first mucolytic is vitamin E.

E37. The composition as in E29 or E30, wherein the first mucolytic is tris(2-carboxyethyl)phosphine hydrochloride.

E38. The composition as in E29 or E30, wherein the first mucolytic is N-butylcysteine.

E39. The composition as in E29 or E30, wherein the first mucolytic is reduced glutathione.

E40. The composition as in E29 or E30, wherein the first mucolytic is a N-derivative of amino acid cysteine.

E41. The composition as in E29 or E30, wherein the first mucolytic is a C-derivative of amino acid cysteine.

E42. The composition as in E29 or E30, wherein the first mucolytic is a di-peptide of cysteine and glutamic acid.

E43. The composition as in E29 or E30, wherein the first mucolytic is a di-peptide of aspartic acid.

E44. The composition as in E29 or E30, wherein the first mucolytic is Ambroxol Hydrochloride.

E45. The composition as in E29 or E30, wherein the first mucolytic is a DNAse.

E46. The composition as in E29 or E30, wherein the first mucolytic is a DNA cleaving agent.

E47. The composition as in any one of E29 to E46, wherein the second mucolytic is 2-mercaptoethane sulfonate (e.g., sodium 2-mercaptoethane sulfonate).

E48. The composition as in any one of E29 to E46, wherein the second mucolytic is N-acetylcysteine.

E49. The composition as in any one of E29 to E46, wherein the second mucolytic is L-α-ureido-mercaptopropionic acid.

E50. The composition as in any one of E29 to E46, wherein the second mucolytic is Bromhexine.

E51. The composition as in any one of E29 to E46, wherein the second mucolytic is ascorbic acid.

E52. The composition as in any one of E29 to E46, wherein the second mucolytic is vitamin E.

E53. The composition as in any one of E29 to E46, wherein the second mucolytic is tris(2-carboxyethyl)phosphine hydrochloride.

E54. The composition as in any one of E29 to E46, wherein the second mucolytic is N-butylcysteine.

E55. The composition as in any one of E29 to E46, wherein the second mucolytic is reduced glutathione.

E56. The composition as in any one of E29 to E46, wherein the second mucolytic is a N-derivative of amino acid cysteine.

E57. The composition as in any one of E29 to E46, wherein the second mucolytic is a C-derivative of amino acid cysteine.

E58. The composition as in any one of E29 to E46, wherein the second mucolytic is a di-peptide of cysteine and glutamic acid.

E59. The composition as in any one of E29 to E46, wherein the second mucolytic is a di-peptide of aspartic acid.

E60. The composition as in any one of E29 to E46, wherein the second mucolytic is Ambroxol Hydrochloride.

E61. The composition as in any one of E29 to E46, wherein the second mucolytic is a DNAse.

E62. The composition as in any one of E29 to E46, wherein the second mucolytic is a DNA cleaving agent.

E63. The composition as in any one of E29 to E34, wherein the first population of inhalable particles are sized to be from about 5 microns to about 49 microns, the second population of inhalable particles are sized to be from about 0.01 microns to about 6 microns, whereby the first population of inhalable particles are configured for predominant absorption at a first target location of the airways of a subject and the second population of inhalable particles are configured for predominant absorption at a second target location of the airways of a subject.

E64. The composition as in E63, wherein the first target location of the airways of the subject is the upper respiratory tract and the second target location of the airways of the subject is the lower respiratory tract.

E65. The composition as in E63 or E64 further comprising additional particles of mucolytics contained within biodegradable encapsulations, the additional particles are sized for predominant absorption at additional locations of the airways upon inhalation of the composition by the subject.

E66. The composition as in any one of E63 to E65, wherein the first biodegradable encapsulation is configured to release the first mucolytic immediately upon inhalation of the composition by the subject, the second biodegradable encapsulation is configured to release the second mucolytic with a predetermined delay, whereby providing an extended time release mucolytic therapy to the subject.

E67. The composition as in E66 further comprising additional particles comprising a mucolytic contained within a biodegradable encapsulation configured to release the mucolytic with additional predetermined delays.

E68. The composition as in any one of E29 to E67, wherein the first inhalable particles and the second inhalable particles are stored in a solution form or a dry powder form.

E69. The composition as in any one of E1 to E68, wherein the first inhalable particles are stored in a solution form.

E70. The composition as in any one of E1 to E68, wherein the first inhalable particles are stored in a dry form.

E71. The composition as in any one of E29 to E70, wherein the second inhalable particles are stored in a solution form.

E72. The composition as in any one of E29 to E70, wherein the second inhalable particles are stored in a dry form.

E73. The composition as in any one of E29 to E72, wherein the dispersing apparatus is configured upon activation to provide each dose of the composition containing from about 5 mg to about 200 milligrams of the first mucolytic.

E74. The composition as in any one of E1 to E45 further comprising one or more short-acting beta-agonist, long-acting beta-agonist, short-acting muscarinic antagonist, long-acting muscarinic antagonist, immunosuppressant, antibiotic, antiviral, antifungal, corticosteroid, or additional anti-infective agent.

E75. The composition as in any one of E1 to E74, wherein the composition comprises an immunosuppressant.

E76. The composition as in E74 or E75, wherein the immunosuppressant is cyclosporin.

E77. The composition as in any one of E1 to E76, wherein the composition comprises an anti-infective agent.

E78. The composition as in E74 or E77, wherein the anti-infective agent is a quinolone.

E79. The composition as in any one of E74, E77, or E78, wherein the anti-infective agent is nalidixic acid.

E80. The composition as in any one of E74, E77, or E78, wherein the anti-infective agent is ciprofloxacin.

E81. The composition as in any one of E74, E77, or E78, wherein the anti-infective agent is ciprofloxacin.

E82. The composition as in any one of E74, E77, or E78, wherein the anti-infective agent is cinoxacin.

E83. The composition as in E74 or E77, wherein the anti-infective agent is a sulfonamide.

E84. The composition as in any one of E74, E77, or E78, wherein the anti-infective agent is sulanilamide.

E85. The composition as in any one of E74, E77, or E78, wherein the anti-infective agent is sulfadiazine.

E86. The composition as in any one of E74, E77, or E78, wherein the anti-infective agent is sulfamethoxazole.

E87. The composition as in any one of E74, E77, or E78, wherein the anti-infective agent is sulfisoxazole.

E88. The composition as in any one of E74, E77, or E78, wherein the anti-infective agent is sulfacetamide.

E89. The composition as in E74 or E77, wherein the anti-infective agent is an aminoglycoside.

E90. The composition as in any one of E74, E77, or E89, wherein the anti-infective agent is streptomycin.

E91. The composition as in any one of E74, E77, or E89, wherein the anti-infective agent is gentamicin.

E92. The composition as in any one of E74, E77, or E89, wherein the anti-infective agent is tobramycin.

E93. The composition as in any one of E74, E77, or E89, wherein the anti-infective agent is amikacin.

E94. The composition as in any one of E74, E77, or E89, wherein the anti-infective agent is netilmicin.

E95. The composition as in any one of E74, E77, or E89, wherein the anti-infective agent is Kanamycin.

E96. The composition as in E74 or E77, wherein the anti-infective agent is a tetracycline.

E97. The composition as in E74, E77, or E96, wherein the anti-infective agent is chlortetracycline.

E98. The composition as in E74, E77, or E96, wherein the anti-infective agent is oxytetracycline.

E99. The composition as in E74, E77, or E96, wherein the anti-infective agent is methacycline.

E100. The composition as in E74, E77, or E96, wherein the anti-infective agent is doxycycline.

E101. The composition as in E74, E77, or E96, wherein the anti-infective agent is minocycline.

E102. The composition as in E74 or E77, wherein the anti-infective agent is a para-aminobenzoic acid.

E103. The composition as in E74 or E77, wherein the anti-infective agent is a diaminopyrimidine.

E104. The composition as in E74, E77, or E103, wherein the anti-infective agent is trimethoprim.

E105. The composition as in E74 or E77, wherein the anti-infective agent is a beta-lactam.

E106. The composition as in any one of E74, E77, or E105, wherein the anti-infective agent is a penicillin.

E107. The composition as in any one of E74, E77, E105, or E106, wherein the anti-infective agent is penicillin G benzathine.

E108. The composition as in any one of E74, E77, E105, or E106, wherein the anti-infective agent is penicillin VK.

E109. The composition as in any one of E74, E77, E105, or E106, wherein the anti-infective agent is ampicillin.

E110. The composition as in any one of E74, E77, E105, or E106, wherein the anti-infective agent is amoxicillin.

E111. The composition as in any one of E74, E77, E105, or E106, wherein the anti-infective agent is bacampicillin.

E112. The composition as in any one of E74, E77, E105, or E106, wherein the anti-infective agent is carbenicillin.

E113. The composition as in any one of E74, E77, E105, or E106, wherein the anti-infective agent is carbenicillin indanyl.

E114. The composition as in any one of E74, E77, E105, or E106, wherein the anti-infective agent is ticarcillin.

E115. The composition as in any one of E74, E77, E105, or E106, wherein the anti-infective agent is azlocillin.

E116. The composition as in any one of E74, E77, E105, or E106, wherein the anti-infective agent is mezlocillin.

E117. The composition as in any one of E74, E77, E105, or E106, wherein the anti-infective agent is piperacillin.

E118. The composition as in any one of E74, E77, E86, or E108, wherein the anti-infective agent is a penicillinase resistant penicillin.

E119. The composition as in any one of E74, E77, E105, E106, or E118, wherein the anti-infective agent is methicillin.

E120. The composition as in any one of E74, E77, E105, E106, or E118, wherein the anti-infective agent is oxacillin.

E121. The composition as in any one of E74, E77, E105, E106, or E118, wherein the anti-infective agent is cloxacillin.

E122. The composition as in any one of E74, E77, E105, E106, or E118, wherein the anti-infective agent is dicloxacillin.

E123. The composition as in any one of E74, E77, E105, E106, or E118, wherein the anti-infective agent is nafcillin.

E124. The composition as in E74 or E77, wherein the anti-infective agent is a cephalosporin.

E125. The composition as in any one of E74, E77, or E124, wherein the anti-infective agent is a first-generation cephalosporin.

E126. The composition as in any one of E74, E77, E124, or E125, wherein the anti-infective agent is cefadroxil.

E127. The composition as in any one of E74, E77, E124, or E125, wherein the anti-infective agent is cephalexin.

E128. The composition as in any one of E74, E77, E124, or E125, wherein the anti-infective agent is cephradine.

E129. The composition as in any one of E74, E77, E124, or E125, wherein the anti-infective agent is cephalothin.

E130. The composition as in any one of E74, E77, E124, or E125, wherein the anti-infective agent is cephapirin.

E131. The composition as in any one of E74, E77, E124, or E125, wherein the anti-infective agent is cefazolin.

E132. The composition as in any one of E46, E49, or E124, wherein the anti-infective agent is a second-generation cephalosporin.

E133. The composition as in any one of E74, E77, E124, or E132, wherein the anti-infective agent is cefaclor.

E134. The composition as in any one of E74, E77, E124, or E132, wherein the anti-infective agent is cefamandole.

E135. The composition as in any one of E74, E77, E124, or E132, wherein the anti-infective agent is cefonicid.

E136. The composition as in any one of E74, E77, E124, or E132, wherein the anti-infective agent is cefoxitin.

E137. The composition as in any one of E74, E77, E124, or E132, wherein the anti-infective agent is cefotetan.

E138. The composition as in any one of E74, E77, E124, or E132, wherein the anti-infective agent is cefuroxime.

E139. The composition as in any one of E74, E77, E124, or E132, wherein the anti-infective agent is aefuroxime axetil.

E140. The composition as in any one of E74, E77, E124, or E132, wherein the anti-infective agent is cefinetazole.

E141. The composition as in any one of E74, E77, E124, or E132, wherein the anti-infective agent is cefprozil.

E142. The composition as in any one of E74, E77, E124, or E132, wherein the anti-infective agent is loracarbef.

E143. The composition as in any one of E74, E77, E124, or E132, wherein the anti-infective agent is ceforanide.

E144. The composition as in any one of E74, E77, or E124, wherein the anti-infective agent is a third-generation cephalosporin.

E145. The composition as in any one of E74, E77, E124, or E144, wherein the anti-infective agent is cefepime.

E146. The composition as in any one of E74, E77, E124, or E144, wherein the anti-infective agent is cefoperaZone.

E147. The composition as in any one of E74, E77, E124, or E144, wherein the anti-infective agent is cefotaxime.

E148. The composition as in any one of E74, E77, E124, or E144, wherein the anti-infective agent is ceftizoxime.

E149. The composition as in any one of E74, E77, E124, or E144, wherein the anti-infective agent is ceftriaxone.

E150. The composition as in any one of E74, E77, E124, or E144, wherein the anti-infective agent is ceftazidime.

E151. The composition as in any one of E74, E77, E124, or E144, wherein the anti-infective agent is cefixime.

E152. The composition as in any one of E74, E77, E124, or E144, wherein the anti-infective agent is cefpodoxime.

E153. The composition as in any one of E74, E77, E124, or E144, wherein the anti-infective agent is ceftibuten.

E154. The composition as in any one of E74, E77, E124, or E144, wherein the anti-infective agent is cefotaxime.

E155. The composition as in any one of E74, E87, or E107, wherein the anti-infective agent is imipenem.

E156. The composition as in any one of E74, E87, or E107, wherein the anti-infective agent is meropenem.

E157. The composition as in any one of E74, E87, or E107, wherein the anti-infective agent is aztreonam.

E158. The composition as in any one of E74, E87, or E107, wherein the anti-infective agent is clavulanic acid.

E159. The composition as in any one of E74, E87, or E107, wherein the anti-infective agent is sulbactam.

E160. The composition as in any one of E74, E87, or E107, wherein the anti-infective agent is tazobactam.

E161. The composition as in E74 or E77, wherein the anti-infective agent is a beta-lactamase inhibitor.

E162. The composition as in any one of E74, E77, or E161, wherein the anti-infective agent is clavulanate.

E163. The composition as in E74 or E77, wherein the anti-infective agent is chloramphenicol.

E164. The composition as in E74 or E77, wherein the anti-infective agent is a macrolide.

E165. The composition as in any one of E74, E77, or E164, wherein the anti-infective agent is erythromycin.

E166. The composition as in any one of E74, E77, or E164, wherein the anti-infective agent is azithromycin.

E167. The composition as in any one of E74, E77, or E164, wherein the anti-infective agent is clarithromycin.

E168. The composition as in E74 or E77, wherein the anti-infective agent is lincomycin.

E169. The composition as in E74 or E77, wherein the anti-infective agent is clindamycin.

E170. The composition as in E74 or E77, wherein the anti-infective agent is spectinomycin.

E171. The composition as in E74 or E77, wherein the anti-infective agent is a polymyxin.

E172. The composition as in any one of E74, E77, or E171, wherein the anti-infective agent is polymyxin A.

E173. The composition as in any one of E74, E77, or E171, wherein the anti-infective agent is polymyxin B.

E174. The composition as in any one of E74, E77, or E171, wherein the anti-infective agent is polymyxin C.

E175. The composition as in any one of E74, E77, or E171, wherein the anti-infective agent is polymyxin D.

E176. The composition as in any one of E74, E77, or E171, wherein the anti-infective agent is polymyxin E.

E177. The composition as in E74 or E77, wherein the anti-infective agent is vancomycin.

E178. The composition as in E74 or E77, wherein the anti-infective agent is bacitracin

E179. The composition as in E74 or E77, wherein the anti-infective agent is isoniazid.

E180. The composition as in E74 or E77, wherein the anti-infective agent is rifampin.

E181. The composition as in E74 or E77, wherein the anti-infective agent is ethambutol.

E182. The composition as in E74 or E77, wherein the anti-infective agent is ethionamide.

E183. The composition as in E74 or E77, wherein the anti-infective agent is aminosalicylic acid.

E184. The composition as in E74 or E77, wherein the anti-infective agent is cycloserine.

E185. The composition as in E74 or E77, wherein the anti-infective agent is capreomycin.

E186. The composition as in E74 or E77, wherein the anti-infective agent is a sulfone.

E187. The composition as in any one of E74, E77, or E186, wherein the anti-infective agent is dapsone.

E188. The composition as in any one of E74, E77, or E186, wherein the anti-infective agent is sulfoxone sodium.

E189. The composition as in E74 or E77, wherein the anti-infective agent is clofazimine.

E190. The composition as in E74 or E77, wherein the anti-infective agent is thalidomide.

E191. The composition as in any one of E1 to E190, wherein the composition comprises an antifungal agent.

E192. The composition as in E74 or E191, wherein the antifungal agent is a polyene.

E193. The composition as in any one of E74, E191, or E192, wherein the antifungal agent is amphotericin B.

E194. The composition as in any one of E74, E191, or E192, wherein the antifungal agent is nystatin.

E195. The composition as in any one of E74, E191, or E192, wherein the antifungal agent is natamycin.

E196. The composition as in E74 or E191, wherein the antifungal agent is flucytosine.

E197. The composition as in E74 or E191, wherein the antifungal agent is an imidazole.

E198. The composition as in any one of E74, E191, or E197, wherein the antifungal agent is miconazole.

E199. The composition as in any one of E74, E191, or E197, wherein the antifungal agent is clotrimazole.

E200. The composition as in any one of E74, E191, or E197, wherein the antifungal agent is econazole.

E201. The composition as in any one of E74, E191, or E197, wherein the antifungal agent is ketoconazole.

E202. The composition as in E74 or E191, wherein the antifungal agent is a triazole.

E203. The composition as in any one of E74, E191, or E202, wherein the antifungal agent is itraconazole.

E204. The composition as in any one of E74, E191, or E202, wherein the antifungal agent is fluconazole.

E205. The composition as in any one of E74, E191, or E202, wherein the antifungal agent is griseofulvin.

E206. The composition as in E74 or E191, wherein the antifungal agent is terconazole.

E207. The composition as in E74 or E191, wherein the antifungal agent is butoconazole.

E208. The composition as in E74 or E191, wherein the antifungal agent is ciclopirox.

E209. The composition as in E74 or E191, wherein the antifungal agent is ciclopirox olamine.

E210. The composition as in E74 or E191, wherein the antifungal agent is haloprogin.

E211. The composition as in E74 or E191, wherein the antifungal agent is tolnaftate.

E212. The composition as in E74 or E191, wherein the antifungal agent is naftifine.

E213. The composition as in E74 or E191, wherein the antifungal agent is terbinafine.

E214. The composition as in any one of E1 to E113, wherein the composition comprises a corticosteroid.

E215. The composition as in E74 or E214, wherein the corticosteroid is flunisolide.

E216. The composition as in E74 or E214, wherein the corticosteroid is fluticasone.

E217. The composition as in E74 or E214, wherein the corticosteroid is fluocortolone.

E218. The composition as in E74 or E214, wherein the corticosteroid is triamcinolone.

E219. The composition as in E74 or E214, wherein the corticosteroid is beclomethasone.

E220. The composition as in E74 or E214, wherein the corticosteroid is budesonide.

E221. The composition as in E74 or E214, wherein the corticosteroid is mometasone.

E222. The composition as in E74 or E214, wherein the corticosteroid is ciclesonide.

E223. The composition as in E74 or E214, wherein the corticosteroid is prednisolone.

E224. The composition as in E74 or E214, wherein the corticosteroid is betamethasone.

E225. The composition as in E74 or E214, wherein the corticosteroid is dexamethasone.

E226. The composition as in E74 or E214, wherein the corticosteroid is hydrocortisone.

E227. The composition as in E74 or E214, wherein the corticosteroid is methylprednisolone.

E228. The composition as in E74 or E214, wherein the corticosteroid is deflazacort.

E229. The composition as in any one of E1 to E228, wherein the composition comprises a short-acting beta-agonist.

E230. The composition as in E74 or E229, wherein the short-acting beta-agonist is bitolterol.

E231. The composition as in E74 or E229, wherein the short-acting beta-agonist is fenoterol.

E232. The composition as in E74 or E229, wherein the short-acting beta-agonist is isoprenaline.

E233. The composition as in E74 or E229, wherein the short-acting beta-agonist is levosalbutamol.

E234. The composition as in E74 or E229, wherein the short-acting beta-agonist is orciprenaline.

E235. The composition as in E74 or E229, wherein the short-acting beta-agonist is pirbuterol.

E236. The composition as in E74 or E229E74 or E229, wherein the short-acting beta-agonist is procaterol.

E237. The composition as in E74 or E229, wherein the short-acting beta-agonist is ritodrine.

E238. The composition as in E74 or E229, wherein the short-acting beta-agonist is salbutamol.

E239. The composition as in E74 or E229, wherein the short-acting beta-agonist is terbutaline.

E240. The composition as in E74 or E229, wherein the short-acting beta-agonist is albuterol.

E241. The composition as in any one of E1 to E240, wherein the composition comprises a long-acting beta-agonist.

E242. The composition as in E74 or E241, wherein the long-acting beta-agonist is formoterol.

E243. The composition as in E74 or E241, wherein the long-acting beta-agonist is bambuterol.

E244. The composition as in E74 or E241, wherein the long-acting beta-agonist is clenbuterol.

E245. The composition as in E74 or E241, wherein the long-acting beta-agonist is formoterol.

E246. The composition as in E74 or E241, wherein the long-acting beta-agonist is salmeterol.

E247. The composition as in E74 or E241, wherein the long-acting beta-agonist is indacaterol.

E248. The composition as in E74 or E241, wherein the long-acting beta-agonist is olodaterol.

E249. The composition as in E74 or E241, wherein the long-acting beta-agonist is vilanterol.

E250. The composition as in any one of E1 to E249, wherein the composition comprises a short-acting muscarinic antagonist.

E251. The composition as in E74 or E250, wherein the short-acting muscarinic antagonist is ipratropium.

E252. The composition as in any one of E1 to E251, wherein the composition comprises a long-acting muscarinic antagonist.

E253. The composition as in E74 or E252, wherein the long-acting muscarinic antagonist is aclidinium.

E254. The composition as in E74 or E252, wherein the long-acting muscarinic antagonist is glycopyrronium.

E255. The composition as in E74 or E252, wherein the long-acting muscarinic antagonist is glycopyrrolate.

E256. The composition as in E74 or E252, wherein the long-acting muscarinic antagonist is tiotropium.

E257. The composition as in E74 or E252, wherein the long-acting muscarinic antagonist is umeclidinium.

E258. The composition as in any one of E1 to E257, wherein the composition comprises a long-acting muscarinic antagonist and a long-acting beta-agonist.

E259. The composition as in E258, wherein the long-acting muscarinic antagonist is umeclidinium and the long-acting beta-agonist is vilanterol.

E260. The composition as in E258, wherein the long-acting muscarinic antagonist is tiotropium and the long-acting beta-agonist is olodaterol.

E261. The composition as in E258, wherein the long-acting muscarinic antagonist is glycopyrrolate and the long-acting beta-agonist is formoterol.

E262. The composition as in E258, wherein the long-acting muscarinic antagonist is glycopyrronium and the long-acting beta-agonist is indacaterol.

E263. The composition as in any one of E1 to E262, wherein the composition comprises a long-acting muscarinic antagonist and a short-acting muscarinic antagonist.

E264. The composition as in any one of E1 to E263, wherein the composition comprises a long-acting muscarinic antagonist, short-acting muscarinic antagonist, long-acting beta-agonist, and a corticosteroid.

E265. The composition as in any one of E1 to E264, wherein the composition comprises an antiviral agent.

E266. The composition as in E74 or E264, wherein the antiviral agent is remdesivir, abacavir, adefovir, delavirdine, descovy, didanosine, doravirine, efavirenz, emtricitabine, entecavir, etravirine, lamivudine, loviride, nevirapine, rilpivirine, stavudine, tenofovir alafenamide, tenofovir disoproxil, zalcitabine, zidovudine, acyclovir, cidofovir, penciclovir, famciclovir, foscarnet, ganciclovir, valganciclovir, idoxuridine, ribavirin, taribavirin, sofosbuvir, telbivudine, trifluridine, valaciclovir, vidarabine, boceprevir, atazanavir, darunavir, fosamprenavir, indinavir, lopinavir, nelfinavir, ritonavir, saquinavir, simeprevir, telaprevir, tipranavir, bictegravir, elvitegravir, dolutegravir, raltegravir, oseltamivir, zanamivir, laninamivir, peramivir, ledipasvir, amprenavir, amantadine, umifenovir, baloxavir marboxil, daclatasvir, docosanol, edoxudine, enfuvirtide, fomivirsen, ibacitabine, ibalizumab, letermovir, maraviroc, metisazone, moroxydine, nexavir, nitazoxanide, pleconaril, rimantadine, tromantadine, or vicriviroc.

E267. The composition as in any one of E1 to E266, wherein the composition comprises liposomes, microspheres, engineered spray-dried particles, or nanoparticles.

E268. The composition as in any one of E1 to E267, wherein the composition comprises nanoparticles.

E269. The composition as in any one of E74 to E268, wherein the first mucolytic, second mucolytic, short-acting beta-agonist, long-acting beta-agonist, short-acting muscarinic antagonist, long-acting muscarinic antagonist, immunosuppressant, antibiotic, antiviral, antifungal, corticosteroid, or additional anti-infective agent compound are encapsulated in nanoparticles.

E270. The composition as in any one of E74 to E269, wherein the first mucolytic is encapsulated in nanoparticles.

E271. The composition as in any one of E74 to E270, wherein the second mucolytic is encapsulated in nanoparticles.

E272. The composition as in any one of E74 to E271, wherein the short-acting beta-agonist is encapsulated in nanoparticles.

E273. The composition as in any one of E74 to E272, wherein the long-acting beta-agonist is encapsulated in nanoparticles.

E274. The composition as in any one of E74 to E273, wherein the short-acting muscarinic antagonist is encapsulated in nanoparticles.

E275. The composition as in any one of E74 to E274, wherein the long-acting muscarinic antagonist is encapsulated in nanoparticles.

E276. The composition as in any one of E74 to E275, wherein the immunosuppressant is encapsulated in nanoparticles.

E277. The composition as in any one of E74 to E276, wherein the antiviral is encapsulated in nanoparticles.

E278. The composition as in any one of E74 to E277, wherein the antifungal is encapsulated in nanoparticles.

E279. The composition as in any one of E74 to E278, wherein the antibiotic is encapsulated in nanoparticles.

E280. The composition as in any one of E74 to E279, wherein the corticosteroid is encapsulated in nanoparticles.

E281. The composition as in any one of E74 to E280, wherein the anti-infective compound is encapsulated in nanoparticles.

E282. The composition as in any one of E1 to E281, wherein the composition comprises microspheres.

E283. The composition as in any one of E74 to E282, wherein the first mucolytic, second mucolytic, short-acting beta-agonist, long-acting beta-agonist, short-acting muscarinic antagonist, long-acting muscarinic antagonist, immunosuppressant, antibiotic, antiviral, antifungal, corticosteroid, or additional anti-infective agent compound are encapsulated in microspheres.

E284. The composition as in any one of E74 to E283, wherein the first mucolytic is encapsulated in microspheres.

E285. The composition as in any one of E74 to E284, wherein the second mucolytic is encapsulated in microspheres.

E286. The composition as in any one of E74 to E285, wherein the short-acting beta-agonist is encapsulated in microspheres.

E287. The composition as in any one of E74 to E286, wherein the long-acting beta-agonist is encapsulated in microspheres.

E288. The composition as in any one of E74 to E287, wherein the short-acting muscarinic antagonist is encapsulated in microspheres.

E289. The composition as in any one of E74 to E288, wherein the long-acting muscarinic antagonist is encapsulated in microspheres.

E290. The composition as in any one of E74 to E289, wherein the immunosuppressant is encapsulated in microspheres.

E291. The composition as in any one of E74 to E290, wherein the antiviral is encapsulated in microspheres.

E292. The composition as in any one of E74 to E291, wherein the antifungal is encapsulated in microspheres.

E293. The composition as in any one of E74 to E292, wherein the antibiotic is encapsulated in microspheres.

E294. The composition as in any one of E74 to E293, wherein the corticosteroid is encapsulated in microspheres.

E295. The composition as in any one of E74 to E294, wherein the anti-infective compound is encapsulated in microspheres.

E296. The composition as in any one of E1 to E295, wherein the composition comprises liposomes.

E297. The composition as in any one of E74 to E296, wherein the first mucolytic, second mucolytic, short-acting beta-agonist, long-acting beta-agonist, short-acting muscarinic antagonist, long-acting muscarinic antagonist, immunosuppressant, antiviral, antifungal, antibiotic, corticosteroid, or anti-infective compound are encapsulated in liposomes.

E298. The composition as in any one of E74 to E297, wherein the first mucolytic is encapsulated in liposomes.

E299. The composition as in any one of E74 to E298, wherein the second mucolytic is encapsulated in liposomes.

E300. The composition as in any one of E74 to E299, wherein the short-acting beta-agonist is encapsulated in liposomes.

E301. The composition as in any one of E74 to E300, wherein the long-acting beta-agonist is encapsulated in liposomes.

E302. The composition as in any one of E74 to E301, wherein the short-acting muscarinic antagonist is encapsulated in liposomes.

E303. The composition as in any one of E74 to E302, wherein the long-acting muscarinic antagonist is encapsulated in liposomes.

E304. The composition as in any one of E74 to E303, wherein the immunosuppressant is encapsulated in liposomes.

E305. The composition as in any one of E74 to E304, wherein the antiviral is encapsulated in liposomes.

E306. The composition as in any one of E74 to E305, wherein the antifungal is encapsulated in liposomes.

E307. The composition as in any one of E74 to E306, wherein the antibiotic is encapsulated in liposomes.

E308. The composition as in any one of E74 to E307, wherein the corticosteroid is encapsulated in liposomes.

E309. The composition as in any one of E74 to E308, wherein the anti-infective compound is encapsulated in liposomes.

E310. The composition as in any one of E1 to E309, wherein the composition comprises engineered spray-dried particles.

E311. The composition as in any one of E74 to E310, wherein the first mucolytic, second mucolytic, short-acting beta-agonist, long-acting beta-agonist, short-acting muscarinic antagonist, long-acting muscarinic antagonist, immunosuppressant, antiviral, antifungal, antibiotic, corticosteroid, or anti-infective compound are engineered spray-dried particles.

E312. The composition as in any one of E74 to E311, wherein the first mucolytic is in the form of engineered spray-dried particles.

E313. The composition as in any one of E74 to E312, wherein the second mucolytic is in the form of engineered spray-dried particles.

E314. The composition as in any one of E46 to E313, wherein the short-acting beta-agonist is in the form of engineered spray-dried particles.

E315. The composition as in any one of E46 to E314, wherein the long-acting beta-agonist is in the form of engineered spray-dried particles.

E316. The composition as in any one of E46 to E315, wherein the short-acting muscarinic antagonist is in the form of engineered spray-dried particles.

E317. The composition as in any one of E46 to E316, wherein the long-acting muscarinic antagonist is in the form of engineered spray-dried particles.

E318. The composition as in any one of E46 to E317, wherein the immunosuppressant is in the form of engineered spray-dried particles.

E319. The composition as in any one of E46 to E318, wherein the antiviral is in the form of engineered spray-dried particles.

E320. The composition as in any one of E46 to E319, wherein the antifungal is in the form of engineered spray-dried particles.

E321. The composition as in any one of E46 to E320, wherein the antibiotic is in the form of engineered spray-dried particles.

E322. The composition as in any one of E46 to E321, wherein the corticosteroid is in the form of engineered spray-dried particles.

E323. The composition as in any one of E46 to E322, wherein the anti-infective compound is in the form of engineered spray-dried particles.

E324. A method for preparing the composition as in any one of E1 to E323, wherein the method comprises micronization, crystallization, freeze drying, spray freeze drying, or spray drying.

E325. The method as in E324, wherein the micronization involves milling, crushing, grinding, or precipitation from supercritical fluids.

E326. The method as in E324 or E325, wherein the micronization involves milling.

E327. The method as in any one of E324 to E326, wherein the micronization involves crushing.

E328. The method as in any one of E324 to E327, wherein the micronization involves grinding.

E329. The method as in any one of E324 to E328, wherein the micronization involves precipitation from supercritical fluids.

E330. The method as in any one of E324 to E329, wherein the composition is mixed with a sugar. E331. The method as in any one of E324 to E330, wherein the sugar comprises lactose.

E332. The method as in any one of E324 to E331, wherein the sugar comprises maltodextrin.

E333. The method as in any one of E324 to E332, wherein the sugar comprises mannitol.

E334. The method as in any one of E324 to E333, wherein the sugar comprises trehalose.

E335. The method as in any one of E324 to E334, wherein the method comprises spray drying.

E336. The method as in any one of E324 to E335, wherein the spray drying inlet temperature is between 80 and 90° C.

E337. The method as in any one of E324 to E336, wherein the spray drying outlet temperature is between 55 and 65° C.

E338. The method as in any one of E324 to E337, wherein the spray drying feed rate is between 1 and 3 g/min.

E339. The method as in any one of E296 to E338, wherein the spray drying pressure is between 3 and 5 bar.

E340. The method as in any one of E296 to E339, wherein the method comprises freeze drying.

E341. The method as in any one of E296 to E340, wherein the method comprises spray freeze drying.

E342. The method as in any one of E296 to E341 further comprising dissolving the composition.

E343. The method as in any one of E296 to E342, wherein the composition is mixed with one or more of sodium alginate, chitosan, trehalose, raffinose, leucine, hydroxypropylmethylcellulose, hydroxypropylbetacyclodextrin or a dispersing agent.

E344. The method as E343, wherein the dispersing agent is Pluronic F-68.

E345. A method of delivering the composition as in any one of E1 to E323 to a subject in need thereof, the method comprising delivering the composition through the nose, mouth, trachea, or bronchia.

E346. The method as in E345, wherein the composition is delivered through the nose.

E347. The method as in E345 or E346, wherein the composition is delivered through the mouth.

E348. The method as in any one of E345 to E347, wherein the composition is delivered through the trachea.

E349. The method as in any one of E345 to E348, wherein the composition is delivered through the bronchia.

E350. The method as in any one of E345 to E349, wherein the composition is delivered via nebulization.

E351. The method as in any one of E345 to E350, wherein the composition is delivered via instillation.

E352. The method as in any one of E345 to E351, wherein the subject in need is diagnosed with a muco-obstructive, lung, sinus, ear, or airway disease.

E353. The method as in any one of E345 to E352, wherein the subject in need is diagnosed with a lung disease.

E354. The method as in any one of E345 to E353, wherein the subject in need is diagnosed with a sinus disease.

E355. The method as in any one of E345 to E354, wherein the subject in need is diagnosed with an ear disease.

E356. The method as in any one of E345 to E355, wherein the subject in need is diagnosed with an airway disease.

E357. The method as in any one of E345 to E356, wherein the subject in need is diagnosed with primary ciliary dyskinesia.

E358. The method as in any one of E345 to E357, wherein the subject in need is diagnosed with cystic fibrosis.

E359. The method as in any one of E345 to E358, wherein the subject in need is diagnosed with sinusitis.

E360. The method as in any one of E345 to E359, wherein the subject in need is diagnosed with rhinosinusitis.

E361. The method as in any one of E345 to E360, wherein the subject in need is diagnosed with bronchiolitis obliterans.

E362. The method as in any one of E345 to E361, wherein the subject in need is diagnosed with emphysema.

E363. The method as in any one of E345 to E362, wherein the subject in need is diagnosed with bronchitis.

E364. The method as in any one of E345 to E363, wherein the subject in need is diagnosed with bronchiectasis.

E365. The method as in any one of E345 to E364, wherein the subject in need is diagnosed with pneumonitis.

E366. The method as in any one of E345 to E365, wherein the subject in need is diagnosed with pneumonia.

E367. The method as in any one of E345 to E366, wherein the subject in need is diagnosed with chronic obstructive pulmonary disease (COPD).

E368. The method as in any one of E345 to E367, wherein the subject in need is diagnosed with chronic obstructive airway disease (COAD).

E369. The method as in any one of E345 to E368, wherein the subject in need is diagnosed with acute respiratory distress syndrome (ARDS).

E370. The method as in any one of E345 to E369, wherein the subject in need is diagnosed with chronic respiratory distress syndrome (CRDS).

E371. The method as in any one of E345 to E370, wherein the subject in need is diagnosed with COVID-19.

E372. A composition comprising spray-dried sodium 2-mercaptoethane sulfonate particles having a median mass aerodynamic diameter between 1 and 7 µm.

E373. The composition as in E372, wherein the median mass aerodynamic diameter is between 3 and 5 µm.

E374. The composition as in E372or E373, wherein the particles comprise between 20 and 60% w/w sodium 2-mercaptoethane sulfonate.

E375. The composition as in any one of E372 to E374, wherein the particles comprise between 30 and 40% w/w sodium 2-mercaptoethane sulfonate.

E376. The composition as in any one of E372 to E375, wherein the particles comprise between 45 and 55% w/w sodium 2-mercaptoethane sulfonate.

E377. The composition as in any one of E372 to E376, wherein the composition comprises between 5 and 200 mg of sodium 2-mercaptoethane sulfonate.

E378. The composition as in any one of E372 to E377, wherein the composition comprises between 5 and 50 mg of sodium 2-mercaptoethane sulfonate.

E379. The composition as in any one of E372 to E378, wherein the composition comprises between 50 and 100 mg of sodium 2-mercaptoethane sulfonate.

E380. The composition as in any one of E372 to E379, wherein the composition comprises between 100 and 200 mg of sodium 2-mercaptoethane sulfonate.

E381. The composition as in any one of E372 to E380, wherein the composition comprises between 5 and 30 mg of sodium 2-mercaptoethane sulfonate.

E382. The composition as in any one of E372 to E381, wherein the composition comprises between 10 and 20 mg of sodium 2-mercaptoethane sulfonate.

E383. The composition as in any one of E372 to E382, wherein the bulk density of the composition is between 0.1 and 5 g/mL.

E384. The composition as in any one of E372 to E383, wherein the bulk density of the composition is between 1 and 4 g/mL.

E385. The composition as in any one of E372 to E384, wherein the bulk density of the composition is between 2 and 4 g/mL.

E386. The composition as in any one of E372 to E385, wherein the bulk density of the composition is between 3 and 4 g/mL.

E387. The composition as in any one of E372 to E386, wherein the bulk density of the composition is between 1 and 4.5 g/mL.

E388. The composition as in any one of E372 to E387, wherein the bulk density of the composition is between 2 and 4.5 g/mL.

E389. The composition as in any one of E372 to E388, wherein the bulk density of the composition is between 3 and 4.5 g/mL.

E390. The composition as in any one of E372 to E389, wherein the bulk density of the composition is between 4 and 4.5 g/mL.

E391. The composition as in any one of E372 to E390, wherein the bulk density of the composition is between 1 and 5 g/mL.

E392. The composition as in any one of E372 to E391, wherein the bulk density of the composition is between 2 and 5 g/mL.

E393. The composition as in any one of E372 to E392, wherein the bulk density of the composition is between 3 and 5 g/mL.

E394. The composition as in any one of E372 to E393, wherein the bulk density of the composition is between 4 and 5 g/mL.

E395. The composition as in any one of E372 to E394, wherein the fine particle fraction of the composition less than or equal to a mass median aerodynamic diameter of 6 µm is 10 and 40%.

E396. The composition as in any one of E372 to E395, wherein the fine particle fraction of the composition less than or equal to a mass median aerodynamic diameter of 6 µm is between 20 and 50%.

E397. The composition as in any one of E372 to E396, wherein the fine particle fraction of the composition less than or equal to a mass median aerodynamic diameter of 6 µm is between 30 and 60%.

E398. The composition as in any one of E372 to E397, wherein the fine particle fraction of the composition less than or equal to a mass median aerodynamic diameter of 6 µm is between 40 and 70%.

E399. The composition as in any one of E372 to E398, wherein the fine particle fraction of the composition less than or equal to a mass median aerodynamic diameter of 6 µm is between 50 and 80%.

E400. The composition as in any one of E372 to E399, wherein the fine particle fraction of the composition less than or equal to a mass median aerodynamic diameter of 6 µm is between 60 and 90%.

E401. The composition as in any one of E372 to E400, wherein the fine particle fraction of the composition less than or equal to a mass median aerodynamic diameter of 6 µm is between 70 and 90%.

E402. The composition as in any one of E372 to E401, wherein the fine particle fraction of the composition less than or equal to a mass median aerodynamic diameter of 5 µm is between 10 and 40%.

E403. The composition as in any one of E372 to E402, wherein the fine particle fraction of the composition less than or equal to a mass median aerodynamic diameter of 5 µm is between 20 and 50%.

E404. The composition as in any one of E372 to E403, wherein the fine particle fraction of the composition less than or equal to a mass median aerodynamic diameter of 5 µm is between 30 and 60%.

E405. The composition as in any one of E372 to E404, wherein the fine particle fraction of the composition less than or equal to a mass median aerodynamic diameter of 5 µm is between 40 and 70%.

E406. The composition as in any one of E372 to E405, wherein the fine particle fraction of the composition less than or equal to a mass median aerodynamic diameter of 5 µm is between 50 and 80%.

E407. The composition as in any one of E372 to E406, wherein the fine particle fraction of the composition less than or equal to a mass median aerodynamic diameter of 5 µm is between 60 and 90%.

E408. The composition as in any one of E372 to E407, wherein the fine particle fraction of the composition less than or equal to a mass median aerodynamic diameter of 5 µm is between 70 and 90%.

E409. The composition as in any one of E372 to E408, wherein the fine particle fraction of the composition less than or equal to a mass median aerodynamic diameter of 4 µm is between 10 and 40%.

E410. The composition as in any one of E372 to E409, wherein the fine particle fraction of the composition less than or equal to a mass median aerodynamic diameter of 4 µm is between 20 and 50%.

E411. The composition as in any one of E372 to E410, wherein the fine particle fraction of the composition less than or equal to a mass median aerodynamic diameter of 4 µm is between 30 and 60%.

E412. The composition as in any one of E372 to E411, wherein the fine particle fraction of the composition less than or equal to a mass median aerodynamic diameter of 4 µm is between 40 and 70%.

E413. The composition as in any one of E372 to E412, wherein the fine particle fraction of the composition less than or equal to a mass median aerodynamic diameter of 4 µm is between 50 and 80%.

E414. The composition as in any one of E372 to E413, wherein the fine particle fraction of the composition less than or equal to a mass median aerodynamic diameter of 4 µm is between 60 and 90%.

E415. The composition as in any one of E372 to E414, wherein the fine particle fraction of the composition less than or equal to a mass median aerodynamic diameter of 4 µm is between 70 and 90%.

E416. The composition as in any one of E372 to E415, further comprising mannitol, calcium chloride, magnesium stearate, sodium acetate, DPPC (dipalmitoylphosphatidylcholine), soy lecithin, egg lecithin, hydrogenated soybean phosphatidylcholine (HSPC), cholesterol, PEG (polyethylene glycol); DSPE (distearoyl-sn-glycero-phosphoethanolamine); DSPC (distearoylphosphatidylcholine); DOPC (dioleoylphosphatidylcholine); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine), triolein, 1,2-distearoyl-sn-glycero-3-phosphoethanolamine sodium salt (MPEG-DSPE), or DPPG (1,2-Dipalmitoyl-sn-glycero-3-phosphorylglycerol sodium salt).

E417. The composition as in any one of E372 to E416, further comprising DSPC or DPPC.

E418. The composition as in any one of E372 to E417, further comprising calcium chloride.

E419. The composition as in any one of E372 to E418, further comprising DNAse

E420. A method of treating a subject diagnosed with a respiratory illness with the composition as in any one of E372 to E419.

E421. The method as in E420, wherein the subject is a mammal, e.g., a human.

E422. The method as in E420 or E421, wherein the subject in need is diagnosed with a muco-obstructive, lung, sinus, ear, or airway disease.

E423. The method as in any one of E420 to E422, wherein the subject in need is diagnosed with a lung disease.

E424. The method as in any one of E420 to E423, wherein the subject in need is diagnosed with a sinus disease.

E425. The method as in any one of E420 to E424, wherein the subject in need is diagnosed with an ear disease.

E426. The method as in any one of E420 to E425, wherein the subject in need is diagnosed with an airway disease.

E427. The method as in any one of E420 to E426, wherein the subject in need is diagnosed with primary ciliary dyskinesia.

E428. The method as in any one of E420 to E427, wherein the subject in need is diagnosed with cystic fibrosis.

E429. The method as in any one of E420 to E428, wherein the subject in need is diagnosed with sinusitis.

E430. The method as in any one of E420 to E429, wherein the subject in need is diagnosed with rhinosinusitis.

E431. The method as in any one of E420 to E430, wherein the subject in need is diagnosed with bronchiolitis obliterans.

E432. The method as in any one of E420 to E431, wherein the subject in need is diagnosed with emphysema.

E433. The method as in any one of E420 to E432, wherein the subject in need is diagnosed with bronchitis.

E434. The method as in any one of E420 to E433, wherein the subject in need is diagnosed with bronchiectasis.

E435. The method as in any one of E420 to E434, wherein the subject in need is diagnosed with pneumonitis.

E436. The method as in any one of E420 to E435, wherein the subject in need is diagnosed with pneumonia.

E437. The method as in any one of E420 to E436, wherein the subject in need is diagnosed with chronic obstructive pulmonary disease (COPD).

E438. The method as in any one of E420 to E437, wherein the subject in need is diagnosed with chronic obstructive airway disease (COAD).

E439. The method as in any one of E420 to E438, wherein the subject in need is diagnosed with acute respiratory distress syndrome (ARDS).

E440. The method as in any one of E420 to E439, wherein the subject in need is diagnosed with chronic respiratory distress syndrome (CRDS).

E441. The method as in any one of E420 to E440, wherein the subject in need is diagnosed with COVID-19.

E442. The method as in any one of E420 to E441, wherein the subject is treated more than once per month.

E443. The method as in any one of E420 to E442, wherein the subject is treated more than twice per month.

E444. The method as in any one of E420 to E443, wherein the subject is treated more than once per week.

E445. The method as in any one of E420 to E444, wherein the subject is treated more than twice per week.

E446. The method as in any one of E420 to E445, wherein the subject is treated more than once per day.

E447. The method as in any one of E420 to E446, wherein the subject is treated more than twice per day.

E448. The method as in any one of E420 to E447, wherein the subject is treated three to five times per day.

E449. The method as in any one of E420 to E448, wherein the subject is treated as an out-patient.

E450. The method as in any one of E420 to E449, wherein the subject is treated with a cotherapy.

E451. The method as in any one of E420 to E450, wherein the fine particle dose delivered to the subject is between 1 and 10 mg.

E452. The method as in any one of E420 to E451, wherein the fine particle dose delivered to the subject is between 1 and 7 mg.

E453. The method as in any one of E420 to E452, wherein the fine particle dose delivered to the subject is between 3 and 6 mg.

E454. The method as in any one of E420 to E453, wherein the subject is treated with between 10 and 40 mg of therapeutic.

E455. The method as in any one of E420 to E454, wherein the subject is treated with between 10 and 25 mg of therapeutic.

E456. The method as in any one of E420 to E455, wherein the subject is treated with between 20 and 40 mg of therapeutic.

E457. A composition comprising spray-dried DNAse particles having a median mass aerodynamic diameter between 1 and 7 µm.

E458. The composition as in E457, wherein the median mass aerodynamic diameter is between 3 and 5 µm.

E459. The composition as in E457 or E458, wherein the particles comprise between 20 and 60% w/w DNAse.

E460. The composition as in any one of E457 to E459, wherein the particles comprise between 30 and 40% w/w DNAse.

E461. The composition as in any one of E457 to E460, wherein the particles comprise between 45 and 55% w/w DNAse.

E462. The composition as in any one of E457 to E461, wherein the composition comprises between 5 and 200 mg of DNAse.

E463. The composition as in any one of E457 to E462, wherein the composition comprises between 5 and 50 mg of DNAse.

E464. The composition as in any one of E457 to E463, wherein the composition comprises between 50 and 100 mg of DNAse.

E465. The composition as in any one of E457 to E464, wherein the composition comprises between 100 and 200 mg of DNAse.

E466. The composition as in any one of E457 to E465, wherein the composition comprises between 5 and 30 mg of DNAse.

E467. The composition as in any one of E457 to E466, wherein the composition comprises between 10 and 20 mg of DNAse.

E468. The composition as in any one of E457 to E467, wherein the bulk density of the composition is between 0.1 and 5 g/mL.

E469. The composition as in any one of E457 to E468, wherein the bulk density of the composition is between 1 and 4 g/mL.

E470. The composition as in any one of E457 to E469, wherein the bulk density of the composition is between 2 and 4 g/mL.

E471. The composition as in any one of E457 to E470, wherein the bulk density of the composition is between 3 and 4 g/mL.

E472. The composition as in any one of E457 to E471, wherein the bulk density of the composition is between 1 and 4.5 g/mL.

E473. The composition as in any one of E457 to E472, wherein the bulk density of the composition is between 2 and 4.5 g/mL.

E474. The composition as in any one of E457 to E473, wherein the bulk density of the composition is between 3 and 4.5 g/mL.

E475. The composition as in any one of E457 to E474, wherein the bulk density of the composition is between 4 and 4.5 g/mL.

E476. The composition as in any one of E457 to E475, wherein the bulk density of the composition is between 1 and 5 g/mL.

E477. The composition as in any one of E457 to E476, wherein the bulk density of the composition is between 2 and 5 g/mL.

E478. The composition as in any one of E457 to E477, wherein the bulk density of the composition is between 3 and 5 g/mL.

E479. The composition as in any one of E457 to E478, wherein the bulk density of the composition is between 4 and 5 g/mL.

E480. The composition as in any one of E457 to E479, wherein the fine particle fraction of the composition less than or equal to a mass median aerodynamic diameter of 6 µm is 10 and 40%.

E481. The composition as in any one of E457 to E480, wherein the fine particle fraction of the composition less than or equal to a mass median aerodynamic diameter of 6 µm is between 20 and 50%.

E482. The composition as in any one of E457 to E481, wherein the fine particle fraction of the composition less than or equal to a mass median aerodynamic diameter of 6 µm is between 30 and 60%.

E483. The composition as in any one of E457 to E482, wherein the fine particle fraction of the composition less than or equal to a mass median aerodynamic diameter of 6 µm is between 40 and 70%.

E484. The composition as in any one of E457 to E483, wherein the fine particle fraction of the composition less than or equal to a mass median aerodynamic diameter of 6 µm is between 50 and 80%.

E485. The composition as in any one of E457 to E484, wherein the fine particle fraction of the composition less than or equal to a mass median aerodynamic diameter of 6 µm is between 60 and 90%.

E486. The composition as in any one of E457 to E485, wherein the fine particle fraction of the composition less than or equal to a mass median aerodynamic diameter of 6 µm is between 70 and 90%.

E487. The composition as in any one of E457 to E486, wherein the fine particle fraction of the composition less than or equal to a mass median aerodynamic diameter of 5 µm is between 10 and 40%.

E488. The composition as in any one of E457 to E487, wherein the fine particle fraction of the composition less than or equal to a mass median aerodynamic diameter of 5 µm is between 20 and 50%.

E489. The composition as in any one of E457 to E488, wherein the fine particle fraction of the composition less than or equal to a mass median aerodynamic diameter of 5 µm is between 30 and 60%.

E490. The composition as in any one of E457 to E489, wherein the fine particle fraction of the composition less than or equal to a mass median aerodynamic diameter of 5 µm is between 40 and 70%.

E491. The composition as in any one of E457 to E490, wherein the fine particle fraction of the composition less than or equal to a mass median aerodynamic diameter of 5 µm is between 50 and 80%.

E492. The composition as in any one of E457 to E491, wherein the fine particle fraction of the composition less than or equal to a mass median aerodynamic diameter of 5 µm is between 60 and 90%.

E493. The composition as in any one of E457 to E492, wherein the fine particle fraction of the composition less than or equal to a mass median aerodynamic diameter of 5 µm is between 70 and 90%.

E494. The composition as in any one of E457 to E493, wherein the fine particle fraction of the composition less than or equal to a mass median aerodynamic diameter of 4 µm is between 10 and 40%.

E495. The composition as in any one of E457 to E494, wherein the fine particle fraction of the composition less than or equal to a mass median aerodynamic diameter of 4 µm is between 20 and 50%.

E496. The composition as in any one of E457 to E495, wherein the fine particle fraction of the composition less than or equal to a mass median aerodynamic diameter of 4 µm is between 30 and 60%.

E497. The composition as in any one of E457 to E496, wherein the fine particle fraction of the composition less than or equal to a mass median aerodynamic diameter of 4 µm is between 40 and 70%.

E498. The composition as in any one of E457 to E497, wherein the fine particle fraction of the composition less than or equal to a mass median aerodynamic diameter of 4 µm is between 50 and 80%.

E499. The composition as in any one of E457 to E498, wherein the fine particle fraction of the composition less than or equal to a mass median aerodynamic diameter of 4 µm is between 60 and 90%.

E500. The composition as in any one of E457 to E499, wherein the fine particle fraction of the composition less than or equal to a mass median aerodynamic diameter of 4 µm is between 70 and 90%.

E501. The composition as in any one of E457 to E500, further comprising mannitol, calcium chloride, magnesium stearate, sodium acetate, DPPC (dipalmitoylphosphatidylcholine), soy lecithin, egg lecithin, hydrogenated soybean phosphatidylcholine (HSPC), cholesterol, PEG (polyethylene glycol); DSPE (distearoyl-sn-glycero-phosphoethanolamine); DSPC (distearoylphosphatidylcholine); DOPC (dioleoylphosphatidylcholine); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine), triolein, 1,2-distearoyl-sn-glycero-3-phosphoethanolamine sodium salt (MPEG-DSPE), or DPPG (1,2-Dipalmitoyl-sn-glycero-3-phosphorylglycerol sodium salt).

E502. The composition as in any one of E457 to E501, further comprising DSPC or DPPC.

E503. The composition as in any one of E457 to E502, further comprising calcium chloride.

E504. The composition as in any one of E457 to E503, further comprising sodium 2-mercaptoethane sulfonate.

E505. The composition as in any one of E457 to E504, wherein the DNAse is rhDNAse.

E506. A method of treating a subject diagnosed with a respiratory illness with the composition as in any one of E457 to E505.

E507. The method as in E506, wherein the subject is a mammal, e.g., a human.

E508. The method as in E506 or E467, wherein the subject in need is diagnosed with a muco-obstructive, lung, sinus, ear, or airway disease.

E509. The method as in any one of E506 to E508, wherein the subject in need is diagnosed with a lung disease.

E510. The method as in any one of E506 to E509, wherein the subject in need is diagnosed with a sinus disease.

E511. The method as in any one of E506 to E510, wherein the subject in need is diagnosed with an ear disease.

E512. The method as in any one of E506 to E511, wherein the subject in need is diagnosed with an airway disease.

E513. The method as in any one of E506 to E512, wherein the subject in need is diagnosed with primary ciliary dyskinesia.

E514. The method as in any one of E506 to E513, wherein the subject in need is diagnosed with cystic fibrosis.

E515. The method as in any one of E506 to E514, wherein the subject in need is diagnosed with sinusitis.

E516. The method as in any one of E506 to E515, wherein the subject in need is diagnosed with rhinosinusitis.

E517. The method as in any one of E506 to E516, wherein the subject in need is diagnosed with bronchiolitis obliterans.

E518. The method as in any one of E506 to E517, wherein the subject in need is diagnosed with emphysema.

E519. The method as in any one of E506 to E518, wherein the subject in need is diagnosed with bronchitis.

E520. The method as in any one of E506 to E519, wherein the subject in need is diagnosed with bronchiectasis.

E521. The method as in any one of E506 to E520, wherein the subject in need is diagnosed with pneumonitis.

E522. The method as in any one of E506 to E521, wherein the subject in need is diagnosed with pneumonia.

E523. The method as in any one of E506 to E522, wherein the subject in need is diagnosed with chronic obstructive pulmonary disease (COPD).

E524. The method as in any one of E506 to E523, wherein the subject in need is diagnosed with chronic obstructive airway disease (COAD).

E525. The method as in any one of E506 to E524, wherein the subject in need is diagnosed with acute respiratory distress syndrome (ARDS).

E526. The method as in any one of E506 to E525, wherein the subject in need is diagnosed with chronic respiratory distress syndrome (CRDS).

E527. The method as in any one of E506 to E526, wherein the subject in need is diagnosed with COVID-19.

E528. The method as in any one of E506 to E527, wherein the subject is treated more than once per month.

E529. The method as in any one of E506 to E528, wherein the subject is treated more than twice per month.

E530. The method as in any one of E506 to E529, wherein the subject is treated more than once per week.

E531. The method as in any one of E506 to E530, wherein the subject is treated more than twice per week.

E532. The method as in any one of E506 to E531, wherein the subject is treated more than once per day.

E533. The method as in any one of E506 to E532, wherein the subject is treated more than twice per day.

E534. The method as in any one of E506 to E533, wherein the subject is treated three to five times per day.

E535. The method as in any one of E506 to E534, wherein the subject is treated as an out-patient.

E536. The method as in any one of E506 to E535, wherein the subject is treated with a cotherapy.

E537. The method as in any one of E506 to E536, wherein the fine particle dose delivered to the subject is between 1 and 10 mg.

E538. The method as in any one of E506 to E537, wherein the fine particle dose delivered to the subject is between 1 and 7 mg.

E539. The method as in any one of E506 to E538, wherein the fine particle dose delivered to the subject is between 3 and 6 mg.

E540. The method as in any one of E506 to E539, wherein the subject is treated with between 10 and 40 mg of therapeutic.

E541. The method as in any one of E506 to E540, wherein the subject is treated with between 10 and 25 mg of therapeutic.

E542. The method as in any one of E506 to E541, wherein the subject is treated with between 20 and 40 mg of therapeutic.

E543. A method for preparing a dry-powder active ingredient, comprising: (i) providing the active ingredient, a first excipient solution, and a second excipient solution; (ii) microfluidizing the first excipient solution to render the first excipient solution translucent; (iii) adding the active ingredient to the second excipient solution; (v) adding the second excipient solution comprising the active ingredient to the first excipient solution to form a feed solution; and (iv) spray-drying the feed solution.

E544. The method of E543, wherein the active ingredient is selected from a group of compounds consisting of: sodium 2-mercaptoethane sulfonate, N-acetylcysteine, L-α-ureido-mercaptopropionic acid, Bromhexine, ascorbic acid, vitamin E, tris(2-carboxyethyl)phosphine hydrochloride, N-butylcysteine, reduced glutathione, N-derivatives and C-derivatives of amino acid cysteine, di-peptide of cysteine and glutamic acid, di-peptide of aspartic acid, Ambroxol Hydrochloride, DNAse, and a DNA cleaving agent.

E545. The method of E543 or E544, wherein the active ingredient is sodium 2-mercaptoethane sulfonate.

E546. The method of E543 or E544, wherein the active ingredient is a DNAse.

E547. The method of E543 or E544, wherein the active ingredient is a DNA cleaving agent.

E548. The method as in any one of E543 to E547, wherein the first excipient is 1,2-distearoyl-sn-glycero-3-phosphocholine (DSPC).

E549. The method as in any one of E543 to E548, wherein the second excipient is calcium chloride.

E550. The method as in any one of E543 to E549, wherein nitrogen is used as a drying gas.

E551. The method as in any one of E543 to E550, wherein the first and/or second excipient solutions comprise de-ionized water.

E552. The method as in any one of E543 to E551, wherein the spray drying inlet temperature is between 50 and 70° C.

E553. The method as in any one of E543 to E552, wherein the feed solution is spray-dried at a rate of 1-3 g/min.

E554. The method as in any one of E543 to E553, wherein spray-drying inlet pressure is between 3-5 bar.

E555. The method as in any one of E543 to E554, wherein the second excipient solution comprises sodium chloride (NaCl).

E556. The method of E555, wherein the pH of the second excipient solution is adjusted with the NaCl to be 6-6.5.

E557. The method as in any one of E543 to E556, wherein the spray drying inlet temperature is between 80 and 90° C.

E558. The method as in any one of E543 to E557, wherein the feed solution is maintained at a temperature between 60 and 70° C.

E559. The method as in any one of E543 to E557, wherein the active ingredient is added dropwise to the second excipient solution.

E560. The method as in any one of E543 to E557, wherein the second excipient solution comprising the active ingredient is added dropwise to the first excipient solution.

### BRIEF DESCRIPTION OF DRAWINGS

The accompanying drawings, which are incorporated herein, form part of the specification. Together with this written description, the drawings further serve to explain the principles of, and to enable a person skilled in the relevant art(s), to make and use the present invention.

FIG. 1 shows an embodiment of a preparation method of dry-powder active ingredient, e.g., DNAse or sodium 2 mercaptoethane sulfonate.

FIG. 2 shows a Buchi B-290 spray-dryer.

FIG. 3 shows an embodiment of a delivery system for the present compositions, a RS01 cDPI, capsule-based dry-powder inhaler.

### DETAILED DESCRIPTION OF THE INVENTION

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, components and/or circuits have not been described in detail in order to avoid unnecessarily obscuring claimed subject matter. The illustrative embodiments described in the detailed description and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

The present invention is aimed to directly address the underlying pathology of a respiratory disease, i.e. mucus plugs, decreased mucociliary clearance and its subsequent consequences, with inhalable particles comprising a biodegradable enclosure containing pharmaceutical compositions, e.g. comprising amino acid derivatives, natural and synthetic peptides, small-molecules, vitamins, and/or proteolytic and DNA cleaving enzymes as identified below. These molecules may be designed and chosen to cleave disulfide and other bonds, increase the local antioxidant concentration/potential, break and disrupt the hydrogen bonds and hydrophobic interaction in aggregated and altered mucus proteins as well as nucleic acid and lipids. Furthermore, the inhaled biodegradable particles may optionally be tailored to contain drugs, molecular entities or other agents - alone or in various combinations of two or more of these functional agents encapsulated within the same enclosure or in separate enclosures of the same or different sizes. These separately formulated enclosures containing different drugs/agents can optionally be administered together or sequentially and at different times to enhance their individual effectiveness in the treatment.

The invention encompasses therapeutic agents and methods for their direct pulmonary administration through any one or more of the routes including the nasal, intratracheal, and bronchial instillation. The particles of the invention may be delivered by means of administering inhalable liposomes, microspheres, engineered spray-dried particles, or nanoparticles directly to lungs and the respiratory tract. Furthermore, the inhalable particles of the invention may be deployed by direct instillation, dry powder inhalation, nebulized-inhalation, or aerosolized inhalation via airways route. The methods for this direct delivery by inhalation or the intranasal route envision the use of a dispersing apparatus such as an capsule-based monodose low-resistance device aerosolized metered dose inhaler, a handheld portable nebulizer, or compressor-nebulizer inhaling device capable of delivering the drug encapsulated particles via the inhaled and/or intranasal route. Furthermore, the particles of the therapeutic agent of the present invention may be stored in a dry powder form (such as capsule, mono, or multiple dose dry powder inhaler) or in a liquid form (such as ampoules for a nebulizer).

In some embodiments, a dispersing apparatus may include a device comprising a compressed/pressurized inhalable aerosol delivery device and optionally equipped with a smart digital measurement capability for monitoring of patient compliance. Such a device may include, such as, but not limited to, pulsating membrane nebulizers, vibrating mesh nebulizers, small volume nebulizers, pressured-metered dose inhalers, mono-dose and multi-dose dry powder inhalers, and similar devices capable of inhaled delivery of dry powder containing the particles of the invention or liquid solutions containing these particles. Such dispersing devices may have one or more separated medicine holding chambers containing the inhaled particles as described below and configured for simultaneous or sequential administration of such particles according to a predetermined schedule.

The methods of the invention contemplate regimens and dosages useful to the treatment of diseases, infections, and disorders described herein. The preferred regimens and dosages may be determined by first identifying non-adverse event level (NOAEL) dose using a single ascending dose study method in healthy volunteers. Further dose ranging studies would be conducted to identify minimum effective dose and frequency that must be lower than upper limit of NOAEL. The methods described herein may be applied for chronic daily use or every other day, or one week on and one week off or 4 weeks on and 4 weeks off depending upon the results of clinical studies in different patient populations. Patients may be treated according to these methods every day from 1X to 3X/day based on their physician’s recommendation For the purposes of this description, the terms “enclosure,” “encapsulation,” “liposome,” “formulation,” and “coating” are used interchangeably to describe a biodegradable particle containing a suitable drug for treatment of airways. The term “biodegradable” is used herein to describe a biocompatible material which breaks down upon contact with the tissues of the airways and releases the drug inside thereof. Release of the active drug from within these enclosures may be an immediate release, a controlled-release, a sustained-release, and/or a time-released process. In addition, various particles may be combined together for a time-release of desired drugs on a predetermined spaced apart schedule.

In some embodiments, exemplary liposome delivery vehicles may include closed vesicular, nanovesicle, colloidal, bilayer structures formed by lipid, phospholipid, sphingolipids, glycolipids, long chain fatty acids, and biologically acceptable surfactants that form liposomes of varying sizes and compositions. While the specific composition may be an inhaled liposomal formulation, encapsulating these molecules, compounds, agents and/or enzymes may serve to create a physiologically compatible drug-delivery vehicle for these drugs/agents.

The inhaled liposomal formulation(s) may be uniquely tailored for and administered directly to the target location of a respiratory site of pathology, thereby reducing systemic drug exposure caused when the drugs or agents are administered intravenously, intraarterially, intramuscularly, or in oral formulations. This in turn may allow for a significant reduction of the dosage of these agents, which in turn may alleviate many of the side effects associated therewith.

The excipients used in the formulations have been classified as GRAS (Generally Recognized As Safe). Some exemplary excipients include mannitol, magnesium stearate, sodium acetate, DPPC, soy lecithin, egg lecithin, hydrogenated soybean phosphatidylcholine [HSPC], cholesterol, PEG (polyethylene glycol); DSPE (distearoyl-sn-glycero-phosphoethanolamine); DSPC (distearoylphosphatidylcholine); DOPC (dioleoylphosphatidylcholine); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine), triolein, 1,2-distearoyl-sn-glycero-3-phosphoethanolamine sodium salt (MPEG-DSPE), and DPPG (1,2-Dipalmitoyl-sn-glycero-3-phosphorylglycerol sodium salt). They are part of lung surfactants and thus compatible with lung environment. An exemplary device compatible with the invention is the Monodose inhaler with size #3 capsule packaged individually. Using this device the drug particles of the invention will reach deep lungs even for the patients that have breathing problem because the device offers both low resistance and works well with a flow rate of 15-20 L/min-100 L/min depending on a patient’s ability to inhale. Particles of the invention are filled into size #3 capsules as desired for potency and fill weight. Exemplary fill weights are less than 40 mg, e.g., less than 30 mg, less than 25 mg, less than 20 mg, less than 15 mg, less than 10 mg, less than 5 mg, or less than 1 mg. The above described may then be inhaled (e.g., using a Plastiape DPI (RS01) or other inhaler or nebulizer) at a flow rate of 15-60 LPM by the patient (slow and deep inhalation).

Liposomes, microspheres, engineered spray-dried particles, and nanoparticles represent unique drug carriers that can site-specifically deliver the drug while protecting it from interaction with environment (blood, metabolism, exposure to air, etc.). As such these vehicles are suitable for protecting the mucolytics or another drug of choice from premature conversion to its metabolites and loss of activity thereby preserving its potency until the drug is released at the target site. Inherent to the structure/formulation of the liposome made of certain phospholipids, surfactants (e.g., DSPC or DPPC), and other excipient molecules. When delivered to the targeted site, these components may further serve to liquefy the mucus by disrupting hydrophobic and hydrophilic interactions within and between these proteins, as well as penetrate the mucus through lipids.

Liposome encapsulation of active pharmaceutical ingredients is a useful method for targeted drug delivery. Liposome formulations can be used to deliver water-soluble pharmaceuticals to locations, and release them at times, unattainable with direct treatment of the water-soluble pharmaceuticals, as demonstrated in U.S. Pat. No. 4,797,285 herein incorporated by reference in its entirety. Intravenous injection is one route of administration useful to the application of liposome encapsulated active pharmaceutical ingredients. Others include intramuscular and subcutaneous injection, which provide timed release into the blood stream as described in U.S. Pat. No. 5,023,087

Microspheres encapsulating active pharmaceutical compounds may comprise polymers (e.g., polyethylene glycol, polylactide, polyhydroxybutyrate, or polycaprolactone), or copolymers (e.g., polyethylene glycol-polylactide block copolymer). An exemplary method to form microspheres involves dissolving the active pharmaceutical ingredient in a non-aqueous solvent, forming an oil-in-water emulsion with the solution, followed by volatilizing the organic solvent. U.S. Pat. No. 7,011,776 demonstrates the use of microspheres and is incorporated herein by reference in its entirety.

Engineered spray-dried particles are formed in a process in which a mixture of active pharmaceutical compound and carrier is injected into a hot gas stream, thereby forming fine droplets. The mixture may be a solution, suspension, slurry, or similar. As the solvent evaporates individual dry particles on the order of single micron diameter are formed. Further details on the methods of formation of these engineered spray-dried particles are demonstrated in s U.S. Pat. No. 6,673,335, herein incorporated in its entirety. Engineered spray-dried particles may comprise materials unknown or known in the art, e.g., amino- and polyamino-acids, proteins, peptides, enzymes, including fragments and recombinant components thereof, as demonstrated in U.S. Pat. No. 5,993,805 incorporated herein by reference in its entirety.

Nanoparticles can be used as drug carriers for targeted delivery or timed-release of active pharmaceutical compounds. Polymer and co-polymer materials are among those used in preparing nanoparticles for encapsulation. An exemplary method in which active pharmaceutical ingredients can be encapsulated in nanoparticles is interfacial polymerization. In interfacial polymerization the active pharmaceutical ingredient and polymerizable monomer are dissolved in a non-aqueous solvent, which is added to an aqueous solvent. Polymerization and encapsulation occur at the organic-aqueous interface. Prominent examples of polymerizable monomers are the alkylcyanoacrylates (where alkyl is e.g., n-butyl, isobutyl, isohexyl, etc.). Nanoparticle encapsulation can be used to deliver small molecules as well as peptide pharmaceuticals. Further methods and compositions are detailed in U.S. Pat. No. 8,3818,208 and 5,641,515, both incorporated by reference.

The microspheres, liposomes, engineered spray-dried particles, and nanoparticles of the invention may be evaluated based on bulk density. Where density of a single solid object, or fluid, is the mass divided by the volume, bulk density is the mass divided by the volume of many particles. The bulk density of a substance is variable depending on the size of individual particles in the sample. Through micronization, spray-drying, or other processes that affect particle size, the bulk density of a sample may change without going through a chemical transformation. The distance through the respiratory tract that a sample may travel depends in part on bulk density. The bulk density of the present invention may vary between 0.1 and 5 g/mL, e.g., between 0.1 and 0.6, 0.2 and 0.7, 0.3 and 0.8, 0.4 and 0.9, 0.5 and 1, 1 and 1.5, 1.5 and 2, 2 and 3, 3 and 4, 4, and 5 g/mL.

The microspheres, liposomes, engineered spray-dried particles, and nanoparticles used may also be evaluated using the mass median aerodynamic diameter (MMAD). Particles of smaller mass median aerodynamic diameter travel further into the airways of a subject. Particles with sufficiently small aerodynamic diameters will permeate the lower respiratory tract. Particles with larger mass median aerodynamic diameters will settle into the upper respiratory tract. The present invention contemplates the use of a range of MMAD for targeted delivery to any portion of the respiratory tract necessary. In preferred embodiments, the MMAD for the particles of the invention are between 0.1 µm and 10 µm, e.g., between 0.1 and 0.2, 0.2 and 0.3, 0.3 and 0.4, 0.4 and 0.5, 0.5 and 0.6, 0.6 and 0.7, 0.7 and 0.8, 0.8 and 0.9, 0.9 and 1, 1 and 1.25, 1.25 and 1.5, 1.5 and 1.75, 1.75 and 2, 2 and 2.25, 2.25 and 2.5, 2.5 and 2.75, 2.75 and 3, 3 and 3.5, 3.5 and 4, 4 and 4.5, 4.5 and 5, 5 and 5.5, 5.5 and 6, 6 and 6.5, 6.5 and 7, 7 and 7.5, 7.5 and 8, 8 and 8.5, 8.5 and 9, 9 and 9.5, 9.5 and 10, 0.1 and 1, 1 and 3, 3 and 5, 5 and 7, or 7 and 10 µm.

Mass median aerodynamic diameter is the apparent aerodynamic particle size (i.e. the particle size of a water droplet falling at the same terminal velocity as the particle of sample being measured. The Mass median aerodynamic diameter (of a population) = geometric particle size (of a population) * square root of the bulk density.

In a preferred embodiment, the mass median aerodynamic diameter of the particle is 3.0 - 4.5 µm

In a preferred embodiment, the particle distribution of various geometric sizes are as follows: 10% of particles e <1.2 µm, 50% of particles between 3-4.5 µm, and 40% of particles between 4.0-7.9 µm.

The microsphere, liposome, engineered spray-dried particle, freeze dried particles and nanoparticle compositions used may be evaluated using the fine particle fraction (FPF). FPF is the fraction of particles in a composition that have a mass median aerodynamic diameter equal to or below the desired a mass median aerodynamic diameter for targeted drug delivery, e.g., 2 µm, 3 µm, 4 µm, 5 µm, 6 µm, or 7 µm. The desired aerodynamic diameter varies according to the target location in the respiratory tract. When the method employed targets the upper respiratory tract the desired aerodynamic diameter will be larger, and when the method employed targets the lower respiratory tract the desired a mass median aerodynamic diameter will be smaller. Acceptable ranges of FPF in a composition are between 10 and 90% of the composition, e.g., between 10 and 20%, 20 and 30%, 30 and 40%, 40 and 50%, 50 and 60%, 60 and 70%, 70 and 80%, 80 and 90%, 10 and 40%, 20 and 50%, 30 and 60%, 40 and 70%, 50 and 80%, 60 and 90% or 70 and 90% of the composition.

The mass in the FPF that is delivered to a subject is the fine particle dose (FPD). Acceptable ranged of FPD in the methods described herein are between 0.1 and 100 mg, e.g., between 0.1 and 1, 1 and 2, 2 and 3, 3 and 4, 4 and 5, 5 and 6, 6, and 7, 7 and 8, 8 and 9, 9 and 10, 1 and 7, 3 and 6, 1 and 10, 10 and 20, 20 and 30, 30 and 40, 40 and 50, or 50 and 100 mg.

The main functional drug to be contained within a biodegradable encapsulation is a mucolytic. Broadly speaking, such mucolytic may include amino-acid derivatives, peptides, peptide analogues, enzymes, and/or small-molecules as active agents, alone or in combination with one or more of the agents. In embodiments, such mucolytic may be selected from the following compounds:
N-acetylcysteine in concentrations from about 5% to about 25%, such as 5%, 10%, 15%, 20%, 25%, or any concentration in between as the invention is not limited in this regard;
2-mercaptoethane sulfonate (e.g., sodium 2-mercaptoethane sulfonate) in concentrations from about 5% to about 25%, such as 5%, 10%, 15%, 20%, 25%, or any concentration in between as the invention is not limited in this regard;
L-α-ureido-mercaptopropionic acid in concentrations from about 50 mg/ml to about 250 mg/ml, such as 50 mg/ml, 100 mg/ml, 150 mg/ml, 200 mg/ml, 250 mg/ml, or any other concentration within this range as the invention is not limited in this regard;
Bromhexine;
ascorbic acid, such as vitamin-C (reduced ascorbic acid or an ascorbate salt thereof);
N-butylcysteine;
reduced glutathione, such as natural tri-peptide glutathione in the reduced form of glutathione;
N-derivatives and C- derivatives of amino acid cysteine;
di-peptide of cysteine and glutamic acid;
di-peptide of aspartic acid;
Ambroxol hydrochloride;
DNAse, such as a recombinant DNAse; and
DNA cleaving agent.

N-acetylcysteine is an amino acid derivative used to treat thick mucus in patients suffering from cystic fibrosis or chronic obstructive pulmonary disease. In the methods described herein N-acetylcysteine may be administered, for example, intravenously, orally, or inhaled. In preferred embodiments, N-acetylcysteine is inhaled.

2-mercaptoethane sulfonate (MESNA) is used to prevent hemorrhagic cystitis in cancer patients, as well as a mucolytic agent. 2-mercaptoethane sulfonate may have any compatible counterion. It may be administered, for example, intravenously, orally, or inhaled. In preferred embodiments, 2-mercaptoethan sulfonate is inhaled.

Bromhexine is a mucolytic used to clear mucus from the respiratory tract. Used in syrup, tablets, and solution bromhexine may be administered orally or intravenously.

Ascorbic acid is an essential nutrient required for the function of several important enzymes required for tissue repair and the immune system.

Ambroxol hydrochloride is a mucolytic targeting clearing mucus in the respiratory tract. Through action as a surfactant ambroxol separates mucus from the bronchial wall clearing the respiratory tract and protecting against secondary infection.

In some embodiments, an active agent may include amino-acid derivatives that are chosen from D and/or L-isomer derivatives according to the formula:

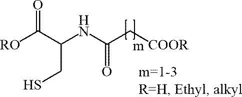

for example:

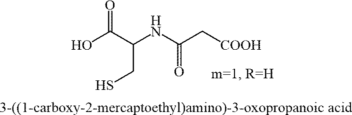

In further embodiments of the invention, the mucolytic may be an amino-acid derivative such as D- and/or L-isomer of the amino acid derivative according to the formula:

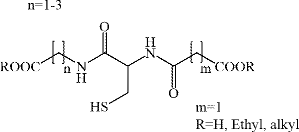

for example:

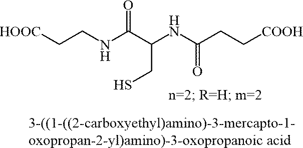

In further embodiments of the invention, the mucolytic may be a peptide or peptide analogue such as D- and/or L-isomer of amino-acids according to the formula:

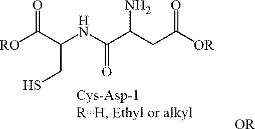

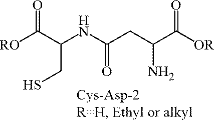

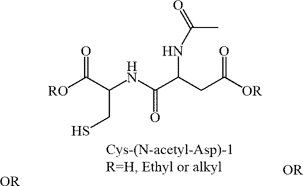

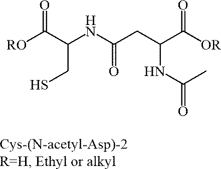

In yet further embodiments of the invention, the mucolytic may be a peptide or a peptide analogue such as D- and/or L-isomer of the amino acids in the peptides according to the formula:

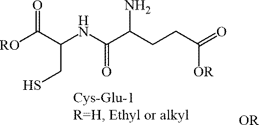

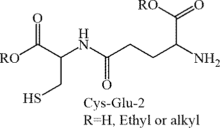

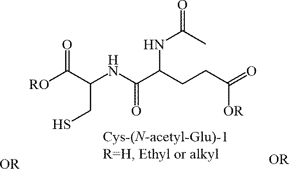

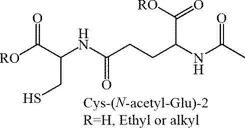

In yet other embodiments, the mucolytic may be vitamin-E or a small-molecule, e.g., sodium 2-mercaptoethane sulfonate or tris(2-carboxyethyl)phosphine hydrochloride.

Since mucus may serve as a form of cover for bacteria, commonly used antibiotics may be unable to reach and attack these bacteria. Breaking the structured mucus and its subsequent liquification may enable an antibiotic to reach the pathogens and bacteria and clear the infection. Therefore, the present invention further includes a simultaneous or sequential administration of inhaled liposomal formulation of a mucolytic as well as an antibiotic, anti-viral, anti-fungal, or another anti-infective compound.

In some embodiments, an anti-infective agent may include quinolones (such as Nalidixic Acid, Cinoxacin, Ciprofloxacin and Norfloxacin and the like), sulfonamides (e.g., Sulfanilamide, Sulfadiazine, Sulfamethoxazole, Sulfisoxazole, Sulfacetamide, and the like), aminoglycosides (e.g., Streptomycin, Gentamicin, Tobramycin, Amikacin, Netilmicin, Kanamycin, and the like), tetracyclines (such as Chlortetracycline, Oxytetracycline, Methacycline, Doxycycline, Minocycline and the like), para-aminobenzoic acid, diaminopyrimidines (such as Trimethoprim, often used in conjunction with Sulfamethoxazole, pyrazinamide, and the like), penicillins (such as Penicillin G, Penicillin V, Ampicillin, Amoxicillin, Bacampicillin, Carbenicillin, Carbenicillin indanyl, Ticarcillin, Azlocillin, Mezlocillin, Piperacillin, and the like), penicillinase resistant penicillin (such as Methicillin, Oxacillin, Cloxacillin, Dicloxacillin, Nafcillin and the like), first-generation cephalosporins (such as Cefadroxil, Cephalexin, Cephradine, Cephalothin, Cephapirin, Cefazolin, and the like), second-generation cephalosporins (such as Cefaclor, Cefamandole, Cefonicid, Cefoxitin, Cefotetan, Cefuroxime, Aefuroxime axetil, Cefinetazole, Cefprozil, Loracarbef, Ceforanide, and the like), third-generation cephalosporins (such as Cefepime, CefoperaZone, Cefotaxime, Ceftizoxime, Ceftriaxone, Ceftazidime, Cefixime, Cefpodoxime, Ceftibuten, and the like), other beta-lactams (such as Imipenem, Meropenem, Aztreonam, Clavulanic acid, Sulbactam, Tazobactam, and the like), beta-lactamase inhibitors (such as Clavulanic acid), Chloramphenicol, macrolides (such as Erythromycin, Azithromycin, Clarithromycin, and the like), Lincomycin, Clindamycin, Spectinomycin, Polymyxin B, polymixins (such as Polymyxin A, B, C or D, E1. Colistin A), or E2, Colistin B or C, and the like) colistin, Vancomycin, Bacitracin, Isoniazid, Rifampin, Ethambutol, Ethionamide, Aminosalicylic Acid, Cycloserine, Capreomycin, sulfones (such as Dapsone, Sulfoxone Sodium, and the like), Clofazimine, Thalidomide, or any other antibacterial agent that can be lipid encapsulated. Antiinfectives can include antifungal agents, including polyene antifungals (such as Amphotericin B, Nystatin, Natamycin, and the like), Flucytosine, imidazole (such as Miconazole, Clotrimazole, Econazole, Ketoconazole, and the like), triazoles (such as Itraconazole, Fluconazole, and the like), Griseofulvin, Terconazole, Butoconazole, Ciclopirox, CiclopiroX Olamine, Haloprogin, Tolnaftate, Naftifine, Terbinafine, or any other antifungals that can be lipid encapsulated or complexed and pharmaceutically acceptable salts thereof and combinations thereof.

In some embodiments, the antiviral may include a reverse-transcriptase inhibitor (e.g, remdesivir, abacavir, adefovir, delavirdine, descovy, didanosine, doravirine, efavirenz, emtricitabine, entecavir, etravirine, lamivudine, loviride, nevirapine, rilpivirine, stavudine, tenofovir alafenamide, tenofovir disoproxil, zalcitabine, or zidovudine), RNA or DNA polymerase inhibitor (e.g., acyclovir, cidofovir, penciclovir, famciclovir, foscarnet, ganciclovir, valganciclovir, idoxuridine, ribavirin, taribavirin, sofosbuvir, telbivudine, trifluridine, valaciclovir, or vidarabine), protease inhibitor (e.g., boceprevir, atazanavir, darunavir, fosamprenavir, indinavir, lopinavir, nelfinavir, ritonavir, saquinavir, simeprevir, telaprevir, or tipranavir), integrase inhibitor (e.g., bictegravir, elvitegravir, dolutegravir, or raltegravir), neuraminidase inhibitor (e.g., oseltamivir, zanamivir, laninamivir, or peramivir), ledipasvir, amprenavir, amantadine, umifenovir, baloxavir marboxil, daclatasvir, docosanol, edoxudine, enfuvirtide, fomivirsen, ibacitabine, ibalizumab, letermovir, maraviroc, metisazone, moroxydine, nexavir, nitazoxanide, pleconaril, rimantadine, tromantadine, or vicriviroc.

In some embodiments, the corticosteroid may include any inhalable steroid, including, but not limited to, flunisolide, fluticasone furoate, fluticasone acetonide, beclomethasone dipropionate, budesonide, mometasone furoate, or ciclesonide.

In some embodiments, the mucolytic may be DNAse, such as a recombinant DNAse, or recombinant human DNAse (rhDNAse). The therapeutics of the present invention may be of any polymorph or formulation amendable to a timed-release, or controlled-release, of inhalable particles. In preferred embodiments, a spray-dried dry powder therapeutic is used. The present invention may use micronized particles, crystalline formulations, or engineered spray-dried particles or solution for their ease of manufacture, slow dissolution rate, and greater stability. Particles may be sized, for example, between 1 and 8 µm, e.g., 1, 2, 3, 4, 5, 6, 7, or 8 µm in diameter. Any method of manufacture for the therapeutic of the invention may be used to give inhalable particles. A preferred embodiment uses micronization to manufacture appropriately sized inhalable particles. Micronization by any method, e.g., milling, crushing, grinding, precipitation from supercritical fluids, and others, may be used to obtain properly sized particles.

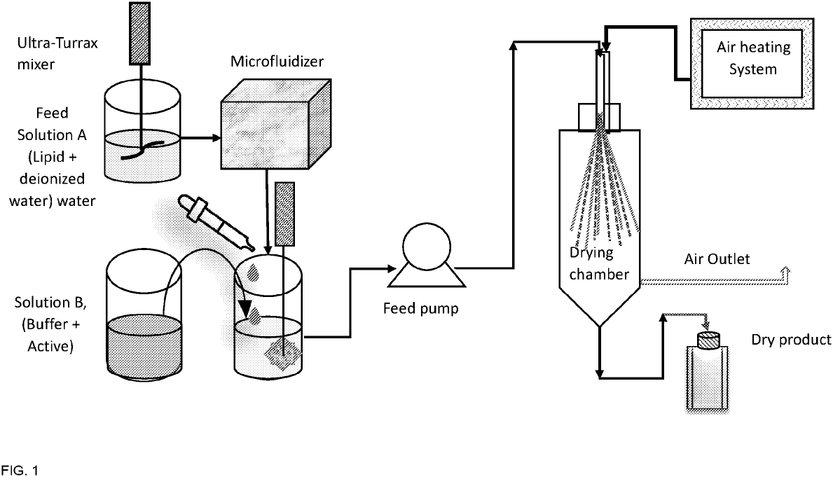

One embodiment of preparing dry-powder DNAse is shown in FIG. 1. The present methods are advantageous, in that the resulting dry-powder DNAse is easier to distribute, store, and administer. The present method may create dry-power DNAse particles sized between 0.01 and 50 microns.

In some embodiments, following preparation, the dry-powder DNAse may be stored at refrigerated conditions, e.g., 2-8° C. In some embodiments, following preparation, the dry-powder DNAse may be stored in reduced humidity environments, e.g., RH <20%.

In embodiments, more than one biodegradable particle may be contained together in a single therapeutic agent. For example, the therapeutic agent of the invention may include a combination of first inhalable particles and second inhalable particles in a dispersing apparatus. Each of the first inhalable particles may in turn comprise a first mucolytic contained within a first biodegradable encapsulation, as described above. Each of the second inhalable particles may in turn comprise the same first or a second mucolytic contained within a second biodegradable encapsulation. In other embodiments, the second particle may contain an anti-infective agent or another drug such as antibiotic, antifungal, or antiviral. In yet another embodiment, the second could be an antihistamine, anti-inflammatory agent, corticosteroid, short-acting beta-agonist (SABA), long-acting beta-agonist (LABA), short-acting muscarinic antagonist (SAMA) and/or long-acting muscarinic antagonist (LAMA). As a result, the first mucolytic and/or the second mucolytic are protected from loss of activity or interaction prior to release from these respective first biodegradable encapsulations or the second biodegradable encapsulations and subsequent absorption at a target location of the airways of the subject.

To assure delivery to more than one target location, first particles may be sized differently than second particles. In embodiments, first particles may be sized from about 4 to about 50 microns for predominant delivery to upper respiratory tract. In embodiments, the size of first particles may be 4-10 microns.

Second particles may be sized to be from about 0.01 to 8 microns to assure predominant delivery at the lower respiratory tract. In embodiments, second particles may be sized to be about 0.01-4 microns, or any size in between.

In other embodiments, drug amount may also vary between first and second particles. Exemplary amount of the mucolytic for the first particles may be from about 5 mg to about 200 mg per dose, and for the second particles from about 10 mg to about 100 mg per dose.

The ratio of first to second inhalable particles in a formulation may very as needed. Exemplary ratios of first to second particles include between 2:1 and 1:2, e.g., 2:1.25, 2:1.5, 2:1.75, 1:1, 1:1.25, 1:1.5, 1:1.75, and 1:2. These ratios are applicable in all embodiments of the invention, regardless what differs between the first and second particle (e.g., size, therapeutic, encapsulation).

In further embodiments, more than two particles may be provided and sized to assure predominant deposition of different particles at different locations along the airways upon inhalation of the therapeutic agent by the subject. In embodiments, three, four or more particle sizes may be provided to allow for a more uniform coverage of the desired portion of the airways with the therapeutic agent of the invention.

In addition to liposomal formulations with lipids, other biodegradable materials or surfactants may be used to create a biodegradable enclosure of the invention. This may be done for a purpose of timed release. In one example, first particles may be designed to release the drug immediately upon contact with the target site, while second particles may be designed to release the drug with a predetermined delay. Combining two or more time-release particles with predetermined times of drug release may be used to create a sustained schedule of drug release following a single or limited number of inhalations. This sustained or controlled release formulation would offer convenient dosing for patients.

Particles may differ between each other not only in size but also in the content of the drug. In embodiments, the same mucolytic may be provided in two or more concentrations within biodegradable particles with different time of release designs. In other embodiments, two different mucolytics may be used to form two or more types of particles of the invention. In yet further embodiments, first particles may contain a mucolytic, while second particles may contain, e.g. an antibiotic or an anti-viral drug.

In embodiments, a total dose of a mucolytic delivered in a single application may vary from about 5 milligrams to about 200 milligrams, such as 5, 10, 20, 30, 40, 50, 75, 100, 125, 150, 175, or 200 milligrams and can be delivered using any of the methods and devices described above.

The present invention as described above may be advantageously used to enable enhanced muco-ciliary clearance (MCC) and directed towards alleviating and treating common fundamental problems and symptoms observed in lung and respiratory airways diseases. A list of conditions and diseases that may benefit from the present invention may include:
primary ciliary dyskinesia (PCD),
cystic fibrosis (CF),
bronchiectasis (BE),
sinusitis,
rhinosinusitis,
bronchiolitis obliterans (BO),
emphysema,
bronchitis,
pneumonia,
pneumonitis,
COPD,
other lung and airways diseases,
other muco-obstructive diseases,
COVID-19,
COAD
Acute Respiratory Distress Syndrome
Chronic Respiratory distress
viral and bacterial infections of lungs, sinus and ear.

Primary ciliary dyskinesia is a recessive genetic disorder affecting the cilia in the respiratory tract. In unaffected patients, epithelial motile cilia vibrate in unison to clear, or move mucus. In primary ciliary dyskinesia the cilia are unable to move in a controlled manner. As a result, the patient is unable to clear mucus in the respiratory tract normally, leading to a buildup of mucus, and potential upper and lower respiratory infections. Other cilia affected can lead to loss of hearing, sense of smell, and taste. Methods of treating PCD may include treatments to ensure adequate muco-ciliary clearance.

Cystic fibrosis, also a recessive genitive disorder, arises from mutations in the cystic fibrosis transmembrane conductance regulator (CFTR) protein. The CFTR protein is a chloride ion channel in epithelial cell membranes and aids in the production of sweat and mucus. Patients affected by CF have improperly thick mucus and suffer from lung infections. Lung transplantation may be required in severe cases. Treatments providing muco-ciliary clearance may be used to combat symptoms of CF.

Bronchiectasis is an obstructive lung disease, acquired from conditions including pneumonia, tuberculosis, cystic fibrosis, and some unknown causes. Sufferers have enlarged airways of the lung, experiencing an inability to properly clear mucus in the airways. Treatments range from physical therapy, to steroids to lung transplantation. Treatments aiding in muco-ciliary clearance may help to prevent infection and secondary conditions.

Sinusitis, or rhinosinusitis, is an inflammation of the mucus membranes in the sinus. The inflammation leads to thickened mucus, headaches, and loss of sense of smell. Sinusitis is an acquired condition, secondary to allergies, and viral and bacterial infections. Tendency to acquire sinusitis is increased in patients with cystic fibrosis and asthma. Treatments are largely focused on symptoms and secondary infections. Treatments focused on muco-ciliary clearance can target the thickened mucus resulting from inflammation.

Obliterative bronchiolitis is an obstructive disorder in the bronchioles due to inflammation. Causes include exposure to toxic fumes, respiratory infections, and complications from lung transplant. Obliterative bronchiolitis is a progressive disease, though treatment with corticosteroids and immunosuppressives may slow the progress. Most patients will succumb to the disease.

Chronic obstructive pulmonary disease, emphysema, and chronic bronchitis refer to a progressive obstructive lung disease. Symptoms include shortness of breath, cough, and sputum production. Causes include exposure to air pollution, tobacco smoking, and some genetic disposition. Symptoms of COPD can be treated with steroids and bronchodilators, and secondary infections treated with antibiotics in severe cases, lung transplantation. Treatments targeted to preventing thickened mucus may aid in clearing the produced sputum.

Pneumonitis is a general inflammation of the lung from exposure to a variety of environmental agents and medical treatments. Untreated, pneumonitis may lead to pulmonary fibrosis. Pneumonia is a related subset of pneumonitis, where pneumonia is a localized condition resulting from infection. Pneumonia is an inflammation of the lung in the alveoli. Bacterial and viral infections are primary causes of pneumonia, however cystic fibrosis, COPD, asthma, and smoking increase susceptibility to pneumonia, among other factors. Pneumonia leads to productive cough, chest pain, and difficulty breathing.

COVID-19 is an infectious respiratory disease leading to acute respiratory distress syndrome (ARDS). COVID-19 is caused by a coronavirus termed severe acute respiratory syndrome coronavirus 2 (SARS-CoV-2), relative of the SARS virus. Since the initial discovery of SARS-CoV-2, the virus spread rapidly across the globe, causing a global pandemic. If an infected host experiences symptoms, it may take days before they manifest. Symptoms include fever, cough, shortness of breath, loss of sense of smell and taste, and in cases with ARDS, multi-organ failure, septic shock, and blood clots, potentially as a result of hypercytokinemia. Hypercytokinemia, or cytokine storm, is an uncontrolled release of cytokines, which are responsible for signaling for inflammation as part of the immune response to infection. The uncontrolled inflammation in the lungs, and other organs, can lead to death. The search for treatments effective in combatting the virus, and the symptoms resulting from infection, has drawn considerable attention globally. In fighting this respiratory virus, treatments aiding in muco-ciliary clearance and maintaining properly thin mucus may help lead to adequate ventilation in affected patients.

Other lung, airways, muco-obstructive diseases, and viral and bacterial infections of the lung, sinus, and ear are also contemplated in the methods of the invention. Among these are included otitis media (e.g., acute otitis media, otitis media with effusion, and chronic suppurative otitis media), tonsillitis, pharyngitis, laryngitis and pleural effusion, as non-limiting examples. The methods of the present invention may be applied to these and other diseases, infections, and disorders to aid in treating causes and symptoms. The causes and symptoms may be in relation to thickened or poorly hydrated mucus, which the methods of the invention may relieve.

The above described diseases, infections, and disorders may be treated with the therapeutic agent of the invention. The methods of the invention, however, also contemplate the combination of the therapeutic agent of the invention with a cotherapy. Common among the above described diseases, infections, and disorders are secondary infections, viral and bacterial, inflammation, or other secondary conditions that may be treated with medicaments not contained in the therapeutic agent of the invention. The cotherapy may be administered in the same in the same formulation with the therapeutic agent of the invention, or in a separate formulation. The cotherapy may be administered before, during, or after the methods described herein. The cotherapy may be administered for a shorter, longer, or same length of time as the therapeutic agent of the invention.

It is contemplated that any embodiment discussed in this specification can be implemented with respect to any method of the invention, and vice versa. It will be also understood that specific embodiments described herein are shown as a way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference. Incorporation by reference is limited such that no subject matter is incorporated that is contrary to the explicit disclosure herein, no claims included in the documents are incorporated by reference herein, and any definitions provided in the documents are not incorporated by reference herein unless expressly included herein.

### Definitions

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps, or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12, 15, 20, or 25%.

The term “dispersibility” or “dispersible” means a dry powder having a moisture content of less than about 10% by weight (%w) water, usually below about 5%w and preferably less than about 3%w; a particle size of about 1.0-5.0 µm mass median diameter (MMD), usually 1.0-4.0 µm MMD, and preferably 1.0-3.0 µm MMD; a delivered dose of about >30%, usually >40%, preferably >50%, and most preferred >60%; and an aerosol particle size distribution of about 1.0-5.0 µm mass median aerodynamic diameter (MMAD), usually 1.5-4.5 µm MMAD, and preferably 1.5-4.0 µm MMAD.

The term “powder” means a composition that consists of finely dispersed solid particles that are free flowing and capable of being readily dispersed in an inhalation device and subsequently inhaled by a subject so that the particles reach the lungs to permit penetration into the alveoli. Thus, the powder is the to be “respirable.” Preferably the averages particle size is less than about 10 microns (µm) in diameter with a relatively uniform spheroidal shape distribution. More preferably the diameter is less than about 7.5 µm and most preferably less than about 5.0 µm. Usually the particle size distribution is between about 0.1 µm and about 5 µm in diameter, particularly about 0.3 µm to about 5 µm.

The term “dry” means that the composition has a moisture content such that the particles are readily dispersible in an inhalation device to form an aerosol. This moisture content is generally below about 10% by weight (%w) water, usually below about 5%w and preferably less than about 3%w.

The term “therapeutically effective amount” is the amount present in the composition that is needed to provide the desired level of drug in the subject to be treated to give the anticipated physiological response. This amount is determined for each drug on a case-by-case basis.

The term “physiologically effective amount” is that amount delivered to a subject to give the desired palliative or curative effect. This amount is specific for each drug and its ultimate approved dosage level.

The term “pharmaceutically acceptable carrier” means that the carrier can be taken into the lungs with no significant adverse toxicological effects on the lungs.

As used herein, the terms “treat,” “treated,” or “treating” mean both therapeutic treatment and prophylactic or preventative measures wherein the object is to prevent or slow down (lessen) an undesired physiological condition, disorder, or disease, or obtain beneficial or desired clinical results. Beneficial or desired clinical results include, but are not limited to, alleviation of symptoms; diminishment of the extent of a condition, disorder, or disease; stabilized (i.e., not worsening) state of condition, disorder, or disease; delay in onset or slowing of condition, disorder, or disease progression; amelioration of the condition, disorder, or disease state or remission (whether partial or total), whether detectable or undetectable; an amelioration of at least one measurable physical parameter, not necessarily discernible by the patient; or enhancement or improvement of condition, disorder, or disease. Treatment includes eliciting a clinically significant response without excessive levels of side effects. Treatment also includes prolonging survival as compared to expected survival if not receiving treatment.

As used herein, the term “microfluidizer” refers to a form of micronization that involves the use high pressure to guide a flow stream through microchannels toward the impingement area, which creates a high shearing action providing a fine emulsion. By using a microfluidizer the distributions of produced particle sizes appear to be narrower and smaller than the products of traditional homogenization,

All of the devices and/or methods disclosed and claimed herein can be made and executed without undue experimentation in light of the present disclosure. While the devices and methods of this invention have been described in terms of preferred embodiments, it will be apparent to those of skill in the art that variations may be applied to the devices and/or methods and in the steps or in the sequence of steps of the method described herein without departing from the concept, spirit and scope of the invention. All such similar substitutes and modifications apparent to those skilled in the art are deemed to be within the spirit, scope, and concept of the invention as defined by the appended claims.

### EXAMPLES

### Example 1. Method of Preparation of Engineered Spray-Dried Particles

A submicron oil-in-water (O/W) emulsion is prepared by high pressure homogenization. The principal excipient in the final spray-dried product and long-chain saturated phospholipids (e.g. distearoylphosphatidylcholine) are incorporated as emulsifiers at the oil/water interface.

The API is mixed dropwise along with matrix forming agents such as sodium alginate (controlled gelation with calcium), chitosan, trehalose, raffinose, leucine, hydroxypropylmethylcellulose, hydroxypropylbetacyclodextrin and/or a dispersing agent such as Pluronic F-68 (a diblock copolymer) into the oil in water emulsion and the resultant mixture is spray dried.

The resulting aqueous dispersion is spray dried. Typical inlet and outlet temperatures on the spray-dryer are 85° C. and 58° C., respectively. In spite of the hot air entering the drying chamber, relatively low temperature conditions prevail throughout the chamber volume. The spray-drying conditions employed will be determined to some extent by the nature of the active agent and excipients to be sprayed. As well, adjustments to the process may be required for some highly lipophilic actives (e.g. steroids) or heat sensitive actives (e.g. peptides and proteins). Typically, the outlet temperature is set below the glass transition or gel to liquid crystal phase transition of the excipients or additives. For this reason, long chain saturated lipids which possess high transition temperatures are preferred.

### Example 2. Characterization of Engineered Spray-Dried Particles

Tap Density. The bulk densities of the spray-dried powders were determined by a tap method (Van Kel Industries Inc., Edison, New Jersey). Bulk powder was added into a tared 1 mL volumetric flask and tapped until the volume remained constant (greater than 10,000 taps).

Particle Size Analysis by Laser Diffraction. A Sympatec laser diffraction analyzer (HELOS H1006, Clausthal-Zellerfeld, Germany) equipped with a RODOS type T4.1 vibrating trough was used to characterize the volume-weighted mean geometric diameter (VMD) of the spray-dried powders. Approximately 1-3 mg of powder was placed in the powder feeder, which was subsequently atomized through a laser beam using 1 bar of air pressure, 60 mbar of vacuum, 70% feed rate and 1.30 mm funnel gap. Data were collected over an interval of 0.4 s, with a 175 µm focal length, triggered at 1% obscuration. Particle size distributions were determined using a Fraünhofer model.

Sedimentation Rates. To assess the improvements in physical stability afforded by homodispersion formation we examined the creaming times (t_{½}) of the hollow porous microspheres, as a function of the volume fraction of blowing agent. The t_{½} was defined as the time required for the suspension to cream or sediment to half-height.

Emitted Dose. Approximately 2 to 4 mg was loaded into No. 4 methylcellulose capsules in a desiccated glovebox (ca. <10%RH). The capsules were actuated for 3 sec. into a 28.3 L min⁻¹ vacuum source. The emitted dose was calculated gravimetrically knowing the capsule weight, total capsule fill weight and actuated capsule weight.

Particle Size of Emulsions. The median diameters of the oil-in-water emulsions containing albuterol sulfate were determined by photosedimentation on a Horiba CAPA-700 (Irvine, California).

Andersen Cascade Impaction. Aerodynamic diameters were determined with an eight-stage Andersen cascade impactor, using an air flow rate of 15-25 liters/minute, and in conformance with the standard USP protocol <601>. Mass median aerodynamic diameter of 2.5-4.5 micron was acceptable.

### Example 3. Micronization of Active Pharmaceutical Ingredients

The API is blended with a surfactant (e.g., DSPC or DPPS) using a turbula blender and then milled using a fluid energy mill. For larger batches a PSD1/Mobile Minor is used targeting larger particles for micronization. Particles are spray dried using ambient air or dry N₂.

### Example 4. Method of Preparation of MESNA/DSPC Engineered Spray-Dry Particles

DSPC was dispersed in DI water with an Ultra-Turrax mixer at 8,000 rpm for 2 to 5 minutes (T = 60-70° C.). The resulting coarse emulsions were homogenized using a microfluidizer at 18,000 psi for 5 passes resulting in a translucent mixture. MESNA and CaCl₂ were each dissolved in separate DI water. The CaCl₂ solution was added dropwise to the MESNA solution with continuous stirring. The combined solution was then added dropwise to the DSPC emulsion with continuous magnetic stirring. This was then spray dried using a Buchi B290 fitted with an inert loop using N₂ as the drying gas. Feed solutions were maintained at 60° C. during spray drying. The following conditions were used: Liquid feed rate: 2 g/min, Pressure: 4 bar, Inlet Temperature: 85° C., Outlet Temperature: 61° C., Aspirator: 100%. The spray dried powders were handled under reduced RH (<20%) overlaid with nitrogen and stored at 2-8° C.

### Example 5. High API Loading Using the Method of Example 4

MESNA/DSPC spray-dry particles were prepared as in Example 4, however having a higher concentration of MESNA.

Two formulations were made, as seen in Table 2 and Table 3. For both, high MESNA concentrations in the range of MESNA: 45-90% w/w; DSPC: 10-50% w/w; and CaCl₂: 3-4% was used with the method of example 4. The feed emulsion was spray dried to produce a fine white powder with low static charge, with a yield of 3.85 g (77%).

TABLE 1

Mean Particle Distribution (n=3)

| Batch | X₁₀ (µm) | X₅₀ (µm) | X₉₀ (µm) | VMD (µm) |
|---|---|---|---|---|
| 151#005A High API Loading: 49.5% w/w Mesna, 47.3% w/w DSPC, 3.2% w/w CaCl2 | 1.04 | 2.45 | 3.99 | 2.50 |
| 151#005B Very High API Loading: 85.8% w/w Mesna, 11.1% w/w DSPC, 3.0% w/w CaCl2. | 1.16 | 2.53 | 4.00 | 2.57 |

TABLE 2

| Samples | Nominal MESNA Loading (% w/w) | % Recovery and activity (vs. Nominal) | Recalculated % w/w MESNA Loading |
|---|---|---|---|
| 151#005A - High API Loading | 49.53 | 111.1 | 55.05 |
| 151#0005B - Very High API Loading | 85.80 | 106.5 | 91.35 |

Particle size analysis was performed on sympatec (aspiros disperser. R3 lens, 1 bar pressure, 30-35mbar depression pressure). Triplicate measurements.
X10 - 1.08 µm
X50 - 3.76 µm
X90 - 7.53 µm
VMD - 4.14 um

Ellman’s assay was used to test for the presence of sulfhydryl groups. Triplicate measurements give a measured API loading of 50.27 %w/w, 101.5% of the loading percentage. 50 mg of spray-dried powders were hand-filled into size 3 HPMC capsules under reduced RH (<20%) and the capsules were stored under N₂.

Emitted dose measurement was taken using a plastiape low-resistance device of the size 3 HPMC capsules. A single actuation was used. The flow rate was 60±3 LPM. The pressure drop across the device was 1.5 kPa. The emitted dose was calculated gravimetrically using the device weight, the capsule weight and the combined device weight with the capsule weight. Measurements were taken in triplicate giving the following results shown in Table 3.

TABLE 3

| Capsule No. | Object | Pre-actuation Weight (g) | Post-actuation Weight (g) | Emitted Dose (g) | % Dose Emitted |
|---|---|---|---|---|---|
| 1 | Device | 10.44579 | 10.44655 | -0.00076 | - |
| | Capsule | 0.09349 | 0.04343 | 0.05008 | 100.1 |
| | Device + Capsule | 1053857 | 10.48894 | 0.04963 | 99.3 |
| 2 | Device | 10.44636 | 10.45186 | -0.0055 | - |
| | Capsule | 0.09986 | 0.04982 | 0.05004 | 100.1 |
| | Device + Capsule | 10.54484 | 10.50171 | 0.04313 | 86.3 |
| 3 | Device | 10.45189 | 10.4609 | -0.00901 | - |
| | Capsule | 0.09727 | 0.04738 | 0.04989 | 99.8 |
| | Device + Capsule | 10.54913 | 10.50883 | 0.0403 | 80.6 |
| Mean | Device + Capsule | | | | 88.7 |

ACI analysis was done using plates coated with 5% w/v Tween 80 in methanol and DI water rinsing solvent, and the same conditions above. A single measurement was made at T=0 and duplicate measurements at T=7 days giving the following results in Table 4.

TABLE 4

| | T=0 | T=7 Days (1) | T=7 Days (2) |
|---|---|---|---|
| Fine Particle Dose mg (≤5.0 µm) | 6.898 | 6.356 | 6.647 |
| Fine Particle Fraction % (≤5.0 µm) | 30.877 | 30.025 | 30.132 |
| MMAD (µm) | 5.213 | 5.081 | 5224 |
| Recovery (%) | 98.21 | 91.95 | 93.72 |

### Example 6. Low API Loading Using the Method of Example 4

Low MESNA concentration (MESNA: 32.95 %w/w, DSPC: 62.8 %w/w, CaCl₂: 4.2 %w/w) was used with the method of Example 4. The feed emulsion was spray dried to produce a fine white powder with low static charge, with a yield of 3.9 g (78%)

Particle size analysis was performed on sympatec (aspiros disperser. R3 lens, 1 bar pressure, 30-35mbar depression pressure). Triplicate measurements;
X10 - 1.07 um
X50 - 3.43 um
X90 - 7.04 um
VMD - 3.80 um

Ellman’s assay was used to test for the presence of sulfhydryl groups. Triplicate measurements give a measured API loading of 33.15 %w/w, 100.6% of the loading percentage. 50 mg of spray-dried powders were hand-filled into size 3 HPMC capsules under reduced RH (<20%) and the capsules were stored under N₂.

Emitted dose measurement was taken using a plastiape low-resistance device of the size 3 HPMC capsules. A single actuation was used. The flow rate was 60±3 LPM. The pressure drop across the device was 1.5 kPa. The emitted dose was calculated gravimetrically using the device weight, the capsule weight and the combined device weight with the capsule weight. Measurements were taken in triplicate giving the following results shown in Table 5.

TABLE 5

| Capsule No. | Object | Pre-actuation Weight (g) | Post-actuation Weight (g) | Emitted Dose (g) | % Dose Emitted |
|---|---|---|---|---|---|
| 1 | Device | 10.4608 | 10.4638 | -0.003 | - |
| | Capsule | 0.09848 | 0.04797 | 0.05051 | 101.0 |
| | Device + Capsule | 10.55908 | 10.5122 | 0.04688 | 93.8 |
| 2 | Device | 10.4637 | 10.46497 | -0.00127 | - |
| | Capsule | 0.09963 | 0.04967 | 0.04998 | 99.9 |
| | Device + Capsule | 10.56293 | 10.51543 | 0.0475 | 95.0 |
| 3 | Device | 10.46485 | 10.46726 | -0.00241 | - |
| | Capsule | 0.09811 | 0.04943 | 0.04868 | 97.4 |
| | Device + Capsule | 10.56285 | 10.5159 | 0.04695 | 93.9 |
| Mean | Device + Capsule | | | | 94.2 |

ACI analysis was done using plates coated with 5% w/v Tween 80 in methanol and DI water rinsing solvent, and the same conditions above. A single measurement was made at T=0 and duplicate measurements at T=7 days giving the following results shown in Table 6.

TABLE 6

| | T=0 | T=7 Days (1) | T=7 Days (2) |
|---|---|---|---|
| Fine Particle Dose mg (≤5.0 µm) | 5.954 | 4.884 | 3.901 |
| Fine Particle Fraction % (≤5.0 µm) | 39.854 | 33.499 | 26.61 |
| MMAD (µm) | 4.453 | 4.936 | 5.492 |
| Recovery (%) | 97.02 | 92.86 | 94.27 |

### Example 7. Production of Spray-Dried Particles

The process of Example 4 was expanded for use with a variety of active drug substances, including DNAse. Spray-dry-powder, inhalable particles comprising active drug substance was manufactured by spray-drying the solution of active drug substance with excipients 1,2-distearoyl-sn-glycero-3-phosphocholine (DSPC) and calcium chloride (CaCl₂) into low density particle that can easily fly at low inspiratory flow rate that is actuated via breath of patients with airway obstruction. The ability to fly at low inspiratory flow rates is important for patients with obstructive airways that have low inspiratory flow rate. An embodiment of the manufacturing method is shown in FIG. 1.

A composition of oil in water emulsion or feed solution (A) was prepared. DSPC was dispersed in de-ionized (DI) water with an Ultra-Turrax mixer at 8,000 rpm for 5 minutes maintaining the temperature at between 60-70° C. The resultant coarse emulsion was microfluidized using an LM10 microfluidizer at 18,000 psi for 5 passes to form a translucent emulsion.

An aqueous feed solution (B) was prepared containing active drug substance. Calcium chloride (CaCl2) and NaCl was dissolved in Deionized water and added dropwise to the active pharmaceutical ingredient solution, such as MESNA or DNAse, with continuous stirring, pH adjusted to 6.3

Feed solution (B) was then added dropwise to the oil-in-water emulsion (A) with continuous magnetic stirring, with temperature of the final feed maintained between 60-70° C.

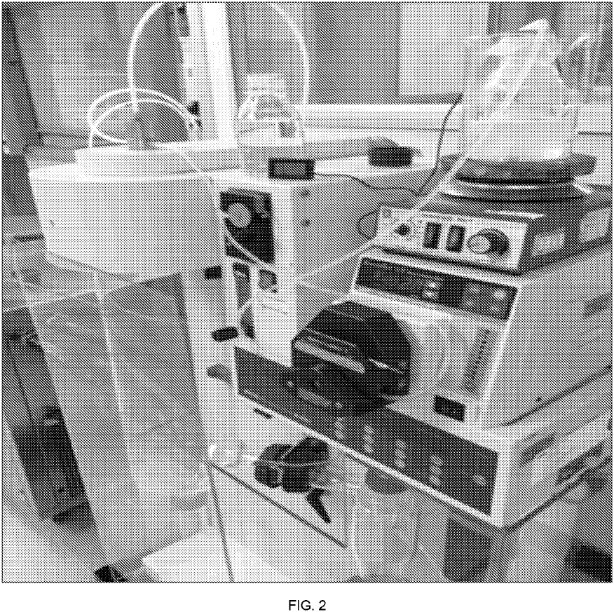

The feed solutions were spray dried using a Buchi B-290 laboratory spray dryer fitted with an inert loop, using nitrogen as the drying gas. A Buchi two fluid spray nozzle (0.7 mm tip diameter) and a standard cyclone were used. A drying outlet temperature of 61° C. was used, with an atomization pressure of 4 bar, and a solution feed rate of 2 g/min. The feed solutions were maintained between 60-70° C., and continually stirred during the spray drying process. A Buchi B-290 spray-dryer is shown in FIG. 2.

The spray-dried powder was collected into amber glass jars, sealed with parafilm and stored at refrigerated conditions (2-80 C). All powder handling was performed in a reduced humidity cabinet (RH≤20%).

Formulation feasibility of non-GMP CIL-06 (DPI rhDNAse) has shown to be stable under accelerated stability conditions of 40° C. and 75% RH for 7 days. Stability after 6 months, one and two years will be tested and confirmed in due course. Emitted dose of CIL-06 formulation was >91% in a simulated administration using a commercially available mono-dose RSO1 inhaler device. It was determined that 50% of the emitted particles are less than 3 µm and optimized mass median aerodynamic diameter (MMAD) was found to be within planned specifications of MMAD < 5 µm for delivery to the site of mucus impaction. The emitted dose was measured using a Dosage Unit Sampling Apparatus (DUSA) tube fitted with a 47 mm glass fiber filter, and the Mass Median Aerodynamic Diameter (MMAD) was measured using an 8-stage Andersen Cascade Impactor (ACI). Particle size analysis of the spray dried powder was carried out using a Sympatec HELOS laser particle size analyzer, fitted with an ASPIROS disperser.

The stability of enzymatic activity of DNase is determined as described by Sinicropi et. al and Xu et al. (Xu, W., Xie, Z., Tong, C. et al. A rapid and sensitive method for kinetic study and activity assay of DNase I in vitro based on a GO-quenched hairpin probe. Anal Bioanal Chem 408, 3801-3809 (2016)).

Samples are reconstituted with 2.5 mL of buffer (containing 25 mM HEPES, 4 mM CaCl2, 4 mM MgCl2, 0.1% BSA, 0.01% thimerosal and 0.05% Tween 20) and diluted to DNase concentrations of 2000 ng/mL, 1000 ng/mL and 500 ng/ml. Substrate comprised of highly polymerized native DNA complexed with methyl green. Hydrolysis of the DNA produced unbound methyl green and a decrease in the absorbance of the solution at 620 nm. By adjusting the time and temperature of the reaction, the assay permits quantification of DNase activity over a wide concentration range (0.4 to 8900 ng/ml). Samples and standards were added to the substrate in microtiter plates and were incubated for 1-24 h at 25-37° C. to achieve the desired assay range. The DNase activity of the samples was interpolated from a standard curve generated with PULMOZYNE®, recombinant human deoxyribonuclease I (rhDNase).

A 96 wells plate were filled with 100 µl of the diluted sample and 100 µL substrate is added. The reaction is performed at room temperature. After 90 minutes the reaction was quenched by adding 50 µL of a solution containing 50 mM EDTA and 50 mM H₂O₂. After another 90 minutes the absorption is measured with a Biorad Microplate Reader Benchmark (Biorad,) at 620 nm. The difference in absorption between 5 and 180 minutes was used to calculate the activity of the samples relative to unprocessed DNase.

### Example 8. Sputum Viscosity and Elasticity Measurements

Sputum is to be collected from healthy, COPD and CF patients. The sputum is to be incubated with quantities of MESNA, DNAse, as well as controls, including PULMOZYME®. At various time points both the elasticity and viscosity of the sputum is to be measured to quantify the mucolytic effects of the present compounds.

### Example 9. DNAse Delivery Devices, Systems, and Methods

Delivery systems for dry-powder DNAse devices, systems, and methods are contemplated.

Dry-powder rhDnase is administered with the RS01 dry powder inhaler (DPI) from Plastiape. The RS01 is a capsule based refillable single-dose Dry Powder Inhaler (DPI). Plastiape has a Type III drug master file (DMF, 17864) filed with the FDA.

RS01 is a convenient and time-efficient breath-actuated portable device. Two versions of the device, low-resistance and high resistance are available. The low resistance device is used so that patients with the lowest inspiratory flow rate can effectively use the inhaler for delivery of rhDnase spray-dried powder. It has been approved for use with dry-powder mannitol in Europe and Australia for several years.

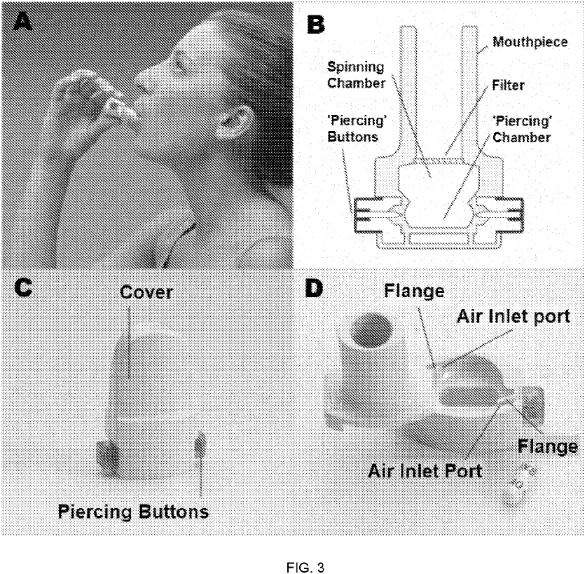

The device may be seen in FIG. 3. The powder evacuates the capsule through the two holes created by the piercing system in the DPI and is inhaled by the patient through the mouthpiece. At the bottom of the mouthpiece, a grid prevents the capsule from reaching the mouthpiece duct.

The inhalers are assembled in ISO 7 clean rooms by high-capacity assembly lines including 100% automated inspection of each critical function. In addition to performance, safety and compliance, the RS01 is lightweight with a minimal number of components. See Table 7 for device specifications.

TABLE 7

Dry Powder Inhaler RS01 Specification

| | | | | | |
|---|---|---|---|---|---|
| General | | Physical Data | | DDS Data | |
| Material | Plastic | Height | 56.3 mm | Capsule | Size 3 |
| Markets | Healthcare | Length | 40.2 mm | Needle | 1×2 |
| Manufacturing Technology | Injection Molding | Width | 23.5 mm | Height 2 Length 2 | 23.5 mm 30 mm |

### OTHER EMBODIMENTS

Various modifications and variations of the described disclosure will be apparent to those skilled in the art without departing from the scope and spirit of the disclosure. Although the disclosure has been described in connection with specific embodiments, it should be understood that the disclosure as claimed should not be unduly limited to such specific embodiments. Indeed, various modifications of the described modes for carrying out the disclosure that are obvious to those skilled in the art are intended to be within the scope of the disclosure.

Other embodiments are in the claims.

1. A composition comprising a population of inhalable particles, wherein the particles comprise a first mucolytic contained in a biodegradable lipid encapsulation, and wherein the composition is for delivery via a dry powder inhaler.

2. The composition of claim 1, wherein the population of inhalable particles are sized to be from about 0.01 microns to about 49 microns, whereby the population of inhalable particles are configured for predominant absorption at a target location of the airways of a subject.

3. The composition of claim 2, wherein the target location of the airways of the subject is the upper respiratory tract or the lower respiratory tract.

4. A composition comprising a first population of inhalable particles and a second population of inhalable particles, wherein the first inhalable particles comprises a first mucolytic contained within a first biodegradable encapsulation, the second population of inhalable particles comprises the first or a second mucolytic contained within a second biodegradable encapsulation.

5. The composition of claim 4, wherein the first mucolytic or the second mucolytic are selected from a group of compounds consisting of: sodium 2-mercaptoethane sulfonate, N-acetylcysteine, L-α-ureido-mercaptopropionic acid, Bromhexine, ascorbic acid, vitamin E, tris(2-carboxyethyl)phosphine hydrochloride, N-butylcysteine, reduced glutathione, N-derivatives and C-derivatives of amino acid cysteine, di-peptide of cysteine and glutamic acid, di-peptide of aspartic acid, Ambroxol Hydrochloride, DNAse, and a DNA cleaving agent.

6. The composition of any of claims 1-5, wherein the first mucolytic is sodium 2-mercaptoethane sulfonate.

7. The composition of any of claims 1-5, wherein the first mucolytic is a DNAse.

8. The composition of any of claims 1-5, wherein the first mucolytic is a DNA cleaving agent.

9. The composition of any one of claims 4 to 8, wherein the second mucolytic is sodium 2-mercaptoethane sulfonate.

10. The composition of any one of claims 4 to 8, wherein the second mucolytic is a DNAse.

11. The composition of any one of claims 4 to 8, wherein the second mucolytic is a DNA cleaving agent.

12. The composition of any one of claims 4 to 11, wherein the first population of inhalable particles are sized to be from about 5 microns to about 49 microns, the second population of inhalable particles are sized to be from about 0.01 microns to about 6 microns, whereby the first population of inhalable particles are configured for predominant absorption at a first target location of the airways of a subject and the second population of inhalable particles are configured for predominant absorption at a second target location of the airways of a subject.

13. The composition of claim 12, wherein the first target location of the airways of the subject is the upper respiratory tract and the second target location of the airways of the subject is the lower respiratory tract.

14. The composition of claim 12 or 13, further comprising additional particles of mucolytics contained within biodegradable encapsulations, the additional particles are sized for predominant absorption at additional locations of the airways upon inhalation of the composition by the subject.

15. The composition of any one of claims 13 to 14, wherein the first biodegradable encapsulation is configured to release the first mucolytic immediately upon inhalation of the composition by the subject, the second biodegradable encapsulation is configured to release the second mucolytic with a predetermined delay, whereby providing an extended time release mucolytic therapy to the subject.

16. The composition of claim 15, further comprising additional particles comprising a mucolytic contained within a biodegradable encapsulation configured to release the mucolytic with additional predetermined delays.

17. The composition of any one of claims 4 to 16, wherein the first inhalable particles and the second inhalable particles are stored in a solution form or a dry powder form.

18. The composition of any one of claims 4 to 17, wherein the dispersing apparatus is configured upon activation to provide each dose of the composition containing from about 5 mg to about 200 milligrams of the first mucolytic.

19. The composition of any one of claims 1 to 18 further comprising one or more short-acting beta-agonist, long-acting beta-agonist, short-acting muscarinic antagonist, long-acting muscarinic antagonist, immunosuppressant, antibiotic, antiviral, antifungal, corticosteroid, or additional anti-infective agent.

20. The composition of any one of claims 1 to 19, wherein the composition comprises an immunosuppressant.

21. The composition of claim 19 or 20, wherein the immunosuppressant is cyclosporin.

22. The composition of any one of claims 1 to 21, wherein the composition comprises an anti-infective agent selected from the group consisting of quinolone, nalidixic acid, ciprofloxacin, cinoxacin, sulfoamide, sulanilamide, sulfadiazine, sulfamethoxazole, sulfisoxazole, aminoglycoside, streptomycin, gentamicin, tobramycin, amikacin, netilmicin, Kanamycin, tetracycline, chlortetracycline, oxytetracycline, methacycline, doxycycline, minocycline, para-aminobenzoic acid, diaminopyrimidine, trimethoprim, beta-lactam, penicillin, penicillin G benzathine, penicillin VK, ampicillin, amoxicillin, bacampicillin, carbenicillin, carbenicillin indanyl, ticarcillin, azlocillin, mezlocillin, piperacillin, penicillinase resistant penicillin, methicillin, oxacillin, cloxacillin, dicloxacillin, nafcillin, cephalosporin, first-generation cephalosporin, cefadroxil, cephalexin, cephradine, cephalothin, cephapirin, cefazolin, second-generation cephalosporin, cefaclor, cefamandole, cefonicid, cefoxitin, cefotetan, cefuroxime, aefuroxime axetil, cefinetazole, cefprozil, loracarbef, ceforanide, third-generation cephalosporin, cefepime, cefoperaZone, cefotaxime, ceftizoxime, ceftriaxone, ceftazidime, cefixime, cefpodoxime, ceftibuten, cefotaxime, imipenem, meropenem, aztreonam, clavulanic acid, sulbactam, tazobactam, beta-lactamase inhibitor, clavulanate, chloramphenicol, macrolide, erythromycin, azithromycin, clarithromycin, lincomycin, clindamycin, spectinomycin, polymyxin, polymyxin A, polymyxin B, polymyxin C, polymyxin D, polymyxin E, vancomycin, bacitracin, isoniazid, rifampin, ethambutol, ethionamide, aminosalicylic acid, cycloserine, capreomycin, sulfone, dapsone, sulfoxone sodium, clofazimine, and thalidomide.

23. The composition of any one of claims 1 to 22, wherein the composition comprises an antifungal agent selected from the group consisting of polyene, amphotericin B, nystatin, natamycin, flucytosine, imidazole, miconazole, clotrimazole, econazole, ketoconazole, triazole, itraconazole, fluconazole, griseofulvin, terconazole, butoconazole, ciclopirox, ciclopirox olamine, haloprogin, tolnaftate, naftifine, and terbinafine.

24. The composition of any one of claims 1 to 23, wherein the composition comprises a corticosteroid selected from the group consisting of flunisolide, fluticasone, fluocortolone, triamcinolone, beclomethasone, budesonide, mometasone, ciclesonide, prednisolone, betamethasone, dexamethasone, hydrocortisone, methylprednisolone, and deflazacort.

25. The composition of any one of claims 1 to 24, wherein the composition comprises a short-acting beta-agonist selected from the group consisting of bitolterol, fenoterol, isoprenaline, levosalbutamol, orciprenaline, pirbuterol, procaterol, ritodrine, salbutamol, terbutaline, albuterol, .

26. The composition of any one of claims 1 to 25, wherein the composition comprises a long-acting beta-agonist selecting from the group consisting of formoterol, bambuterol, clenbuterol, formoterol, salmeterol, indacaterol, olodaterol, and vilanterol.

27. The composition of any one of claims 1 to 26, wherein the composition comprises a short-acting muscarinic antagonist.

28. The composition of claim 19 or 27, wherein the short-acting muscarinic antagonist is ipratropium.

29. The composition of any one of claims 1 to 28, wherein the composition comprises a long-acting muscarinic antagonist selected from the group consisting of aclidinium, glycopyrronium, glycopyrrolate, tiotropium, and umeclidinium.

30. The composition of any one of claims 1 to 29, wherein the composition comprises a long-acting muscarinic antagonist and a long-acting beta-agonist.

31. The composition of claim 30, wherein the long-acting muscarinic antagonist is umeclidinium and the long-acting beta-agonist is vilanterol.

32. The composition of claim 30, wherein the long-acting muscarinic antagonist is tiotropium and the long-acting beta-agonist is olodaterol.

33. The composition of claim 30, wherein the long-acting muscarinic antagonist is glycopyrrolate and the long-acting beta-agonist is formoterol.

34. The composition of claim 30, wherein the long-acting muscarinic antagonist is glycopyrronium and the long-acting beta-agonist is indacaterol.

35. The composition of any one of claims 1 to 34, wherein the composition comprises a long-acting muscarinic antagonist and a short-acting muscarinic antagonist.

36. The composition of any one of claims 1 to 35, wherein the composition comprises a long-acting muscarinic antagonist, short-acting muscarinic antagonist, long-acting beta-agonist, and a corticosteroid.

37. The composition of claim 19 or 36, wherein the composition comprises an antiviral agent selected from the group consisting of remdesivir, abacavir, adefovir, delavirdine, descovy, didanosine, doravirine, efavirenz, emtricitabine, entecavir, etravirine, lamivudine, loviride, nevirapine, rilpivirine, stavudine, tenofovir alafenamide, tenofovir disoproxil, zalcitabine, zidovudine, acyclovir, cidofovir, penciclovir, famciclovir, foscarnet, ganciclovir, valganciclovir, idoxuridine, ribavirin, taribavirin, sofosbuvir, telbivudine, trifluridine, valaciclovir, vidarabine, boceprevir, atazanavir, darunavir, fosamprenavir, indinavir, lopinavir, nelfinavir, ritonavir, saquinavir, simeprevir, telaprevir, tipranavir, bictegravir, elvitegravir, dolutegravir, raltegravir, oseltamivir, zanamivir, laninamivir, peramivir, ledipasvir, amprenavir, amantadine, umifenovir, baloxavir marboxil, daclatasvir, docosanol, edoxudine, enfuvirtide, fomivirsen, ibacitabine, ibalizumab, letermovir, maraviroc, metisazone, moroxydine, nexavir, nitazoxanide, pleconaril, rimantadine, tromantadine, and vicriviroc.

38. The composition of any one of claims 1 to 37, wherein the composition comprises liposomes, microspheres, engineered spray-dried particles, or nanoparticles.

39. The composition of any one of claims 19 to 38, wherein the first mucolytic, second mucolytic, short-acting beta-agonist, long-acting beta-agonist, short-acting muscarinic antagonist, long-acting muscarinic antagonist, immunosuppressant, antibiotic, antiviral, antifungal, corticosteroid, and/or additional anti-infective agent compound are encapsulated in nanoparticles.

40. The composition of any one of claims 1 to 39, wherein the composition comprises microspheres.

41. The composition of any one of claims 19 to 40, wherein the first mucolytic, second mucolytic, short-acting beta-agonist, long-acting beta-agonist, short-acting muscarinic antagonist, long-acting muscarinic antagonist, immunosuppressant, antibiotic, antiviral, antifungal, corticosteroid, and/or additional anti-infective agent compound are encapsulated in microspheres.

42. The composition of any one of claims 1 to 41, wherein the composition comprises liposomes.

43. The composition of any one of claims 19 to 42, wherein the first mucolytic, second mucolytic, short-acting beta-agonist, long-acting beta-agonist, short-acting muscarinic antagonist, long-acting muscarinic antagonist, immunosuppressant, antiviral, antifungal, antibiotic, corticosteroid, and/or anti-infective compound are encapsulated in liposomes.

44. The composition of any one of claims 1 to 43, wherein the composition comprises engineered spray-dried particles.

45. The composition of any one of claims 19 to 44, wherein the first mucolytic, second mucolytic, short-acting beta-agonist, long-acting beta-agonist, short-acting muscarinic antagonist, long-acting muscarinic antagonist, immunosuppressant, antiviral, antifungal, antibiotic, corticosteroid, and/or anti-infective compound are engineered spray-dried particles.

46. A method for preparing the composition of any one of claims 1 to 45, wherein the method comprises micronization, crystallization, freeze drying, spray freeze drying, or spray drying.

47. The method of claim 46, wherein the micronization involves milling, crushing, grinding, or precipitation from supercritical fluids.

48. The method of any one of claims 46 or 47, wherein the composition is mixed with a sugar selected from the group consisting of lactose, maltodextrin, mannitol, and trehalose.

49. The method of any one of claims 46 to 48, wherein the method comprises spray drying.

50. The method of any one of claims 46 to 49, wherein the spray drying inlet temperature is between 80 and 90° C.

51. The method of any one of claims 46 to 50, wherein the spray drying outlet temperature is between 55 and 65° C.

52. The method of any one of claims 46 to 51, wherein the spray drying feed rate is between 1 and 3 g/min.

53. The method of any one of claims 46 to 52, wherein the spray drying pressure is between 3 and 5 bar.

54. The method of any one of claims 46 to 53, wherein the method comprises freeze drying.

55. The method of any one of claims 46 to 54, wherein the method comprises spray freeze drying.

56. The method of any one of claims 46 to 55 further comprising dissolving the composition.

57. The method of any one of claims 46 to 56, wherein the composition is mixed with one or more of sodium alginate, chitosan, trehalose, raffinose, leucine, hydroxypropylmethylcellulose, hydroxypropylbetacyclodextrin or a dispersing agent.

58. The method as 57, wherein the dispersing agent is Pluronic F-68.

59. A method of delivering the composition of any one of claims 1 to 45 to a subject in need thereof, the method comprising delivering the composition through the nose, mouth, trachea, or bronchia.

60. The method of claim 59, wherein the composition is delivered through the nose, mouth, trachea, or bronchia.

61. The method of any one of claims 59 or 60, wherein the composition is delivered via nebulization or instillation.

62. The method of any one of claims 59 to 61, wherein the subject in need is diagnosed with a muco-obstructive, lung, sinus, ear, or airway disease.

63. The method of any one of claims 59 to 62, wherein the subject in need is diagnosed with primary ciliary dyskinesia, cystic fibrosis, sinusitis, rhinosinusitis, bronchiolitis obliterans, emphysema, bronchitis, bronchiectasis, pneumonitis, pneumonia, chronic obstructive pulmonary disease (COPD), chronic obstructive airway disease (COAD), acute respiratory distress syndrome (ARDS), chronic respiratory distress syndrome (CRDS), or COVID-19.

64. A composition comprising spray-dried sodium 2-mercaptoethane sulfonate particles having a median mass aerodynamic diameter between 1 and 7 µm.

65. The composition of claim 64, wherein the median mass aerodynamic diameter is between 3 and 5 µm.

66. The composition of claim 64 or 65, wherein the particles comprise between 20 and 60% w/w sodium 2-mercaptoethane sulfonate.

67. The composition of any one of claims 64 to 66, wherein the composition comprises between 5 and 200 mg of sodium 2-mercaptoethane sulfonate.

68. The composition of any one of claims 64 to 67, wherein the bulk density of the composition is between 0.1 and 5 g/mL.

69. The composition of any one of claims 64 to 68, wherein the fine particle fraction of the composition less than or equal to a mass median aerodynamic diameter of 6 µm is between 10 and 90%.

70. The composition of any one of claims 64 to 69, wherein the fine particle fraction of the composition less than or equal to a mass median aerodynamic diameter of 5 µm is between 10 and 90%.

71. The composition of any one of claims 64 to 70, wherein the fine particle fraction of the composition less than or equal to a mass median aerodynamic diameter of 4 µm is between 10 and 90%.

72. The composition of any one of claims 64 to 71, further comprising mannitol, calcium chloride, magnesium stearate, sodium acetate, DPPC (dipalmitoylphosphatidylcholine), soy lecithin, egg lecithin, hydrogenated soybean phosphatidylcholine (HSPC), cholesterol, PEG (polyethylene glycol); DSPE (distearoyl-sn-glycero-phosphoethanolamine); DSPC (distearoylphosphatidylcholine); DOPC (dioleoylphosphatidylcholine); EPC (egg phosphatidylcholine); DOPS (dioleoylphosphatidylserine); POPC (palmitoyloleoylphosphatidylcholine); SM (sphingomyelin); MPEG (methoxy polyethylene glycol); DMPC (dimyristoyl phosphatidylcholine); DMPG (dimyristoyl phosphatidylglycerol); DSPG (distearoylphosphatidylglycerol); DEPC (dierucoylphosphatidylcholine); DOPE (dioleoly-sn-glycero-phophoethanolamine), triolein, 1,2-distearoyl-sn-glycero-3-phosphoethanolamine sodium salt (MPEG-DSPE), or DPPG (1,2-Dipalmitoyl-sn-glycero-3-phosphorylglycerol sodium salt).

73. A method of treating a subject diagnosed with a respiratory illness with the composition of any one of claims 64-72.

74. The method of claim 73, wherein the subject is a mammal, e.g., a human.

75. The method of claim 73 or 67, wherein the subject in need is diagnosed with a muco-obstructive, lung, sinus, ear, or airway disease.

76. The method of any one of claims 73 to 75, wherein the subject in need is diagnosed with primary ciliary dyskinesia, cystic fibrosis, sinusitis, rhinosinusitis, bronchiolitis obliterans, emphysema, bronchitis, bronchiectasis, pneumonitis, pneumonia, COPD, COAD, ARDS, CRDS, or COVID-19.

77. The method of any one of claims 73 to 76, wherein the subject is treated more than once per month.

78. The method of any one of claims 73 to 77, wherein the subject is treated as an out-patient.

79. The method of any one of claims 73 to 78, wherein the subject is treated with a cotherapy.

80. The method of any one of claims 73 to 79, wherein the fine particle dose delivered to the subject is between 0.1 and 200 mg.

81. The method of any one of claims 73 to 80, wherein the subject is treated with between 0.1 and 200 mg of therapeutic.

82. A method for preparing a dry-powder active ingredient, comprising:
(i) providing the active ingredient, a first excipient solution, and a second excipient solution;
(ii) microfluidizing the first excipient solution to render the first excipient solution translucent;
(iii) adding the active ingredient to the second excipient solution;
(v) adding the second excipient solution comprising the active ingredient to the first excipient solution to form a feed solution; and
(iv) spray-drying the feed solution.

83. The method of claim 82, wherein the active ingredient is selected from a group of compounds consisting of: sodium 2-mercaptoethane sulfonate, N-acetylcysteine, L-α-ureido-mercaptopropionic acid, Bromhexine, ascorbic acid, vitamin E, tris(2-carboxyethyl)phosphine hydrochloride, N-butylcysteine, reduced glutathione, N-derivatives and C-derivatives of amino acid cysteine, di-peptide of cysteine and glutamic acid, di-peptide of aspartic acid, Ambroxol Hydrochloride, DNAse, and a DNA cleaving agent.

84. The method of claim 82 or 83, wherein the active ingredient is sodium 2-mercaptoethane sulfonate.

85. The method of claim 82 or 83, wherein the active ingredient is a DNAse.

86. The method of claim 82 or 83, wherein the active ingredient is a DNA cleaving agent.

87. The method of any of claims 82-86, wherein the first excipient is 1,2-distearoyl-sn-glycero-3-phosphocholine (DSPC).

88. The method of any of claims 82-87, wherein the second excipient is calcium chloride.

89. The method of any of claims 82-88, wherein nitrogen is used as a drying gas.

90. The method of any of claims 82-89, wherein the first and/or second excipient solutions comprise de-ionized water.

91. The method of any of claims 82-90, wherein the spray drying inlet temperature is between 50 and 70° C.

92. The method of any of claims 82-91, wherein the feed solution is spray-dried at a rate of 1-3 g/min.

93. The method of any of claims 82-92, wherein spray-drying inlet pressure is between 3-5 bar.

94. The method of any of claims 82-93, wherein the second excipient solution comprises sodium chloride (NaCl).

95. The method of claim 94, wherein the pH of the second excipient solution is adjusted with the NaCl to be 6-6.5.

96. The method of any of claims 82-95, wherein the spray drying inlet temperature is between 80 and 90° C.

97. The method of any of claims 82-96, wherein the feed solution is maintained at a temperature between 60 and 70° C.
